(19) United States
(12) Patent Application Publication
(10) Pub. No.: US 2025/0132058 A1
(43) Pub. Date: Apr. 24, 2025

(54) Systems and Methods for Adaptive Electronic Medical Record Filtering Leveraging Dynamic Parsing Configuration

(71) Applicant: egnite, Inc., Aliso Viejo, CA (US)

(72) Inventors: Kahla Verhoef, San Clemente, CA (US); Hunter Nance, Newburgh, IN (US)

(73) Assignee: egnite, Inc., Aliso Viejo, CA (US)

(21) Appl. No.: 18/781,146

(22) Filed: Jul. 23, 2024

Related U.S. Application Data
(60) Provisional application No. 63/545,487, filed on Oct. 24, 2023.

(51) Int. Cl. G16H 70/20; G06F 40/205; G16H 10/60; G16H 20/40; G16H 50/70

(52) U.S. Cl. CPC G16H 70/20; G06F 40/205; G16H 10/60; G16H 20/40; G16H 50/70

(57) ABSTRACT

Systems and methods of the present disclosure are configured to determine relevance associated with one or more diseases, conditions and treatments. Based on condition-related criteria and condition-related criteria logic rules derived therefrom, each cardiac patient may be assessed for the relevance of cardiac treatments based on each cardiac patient's EHR data. As such, the condition-related criteria logic rules encode criteria for the applicability of each cardiac treatment based on patient health data, tests, metrics or other factors or any combination thereof. One or more parsers are configured according to the condition-related criteria logic rules to determine a relevance indicator of each cardiac treatment to each cardiac patient. Based on the relevance indicators, a patient list for each cardiac treatment may be updated with the cardiac patients for which the cardiac treatment is relevant.

### RELATED APPLICATIONS(S)

This application claims priority to and the benefit of U.S. Provisional Patent Application No. 63/545,487, filed Oct. 24, 2023, the contents of which is incorporated herein by reference in its entirety.

### FIELD OF TECHNOLOGY

The present disclosure generally relates to computer-based platforms and/or systems configured for adaptive electronic medical record filtering leveraging dynamic parsing configuration(s), such as to generate lists of relevant patients that dynamically update in response to changes in medical records.

### BACKGROUND OF TECHNOLOGY

Gaps in the healthcare system may unintentionally delay care. Across the US, heart programs may fail to identify patients who meet guideline criteria for evaluation and/or intervention, leaving their underlying condition untreated. Additionally, patients may suffer unnecessarily with a risk of worse outcomes due to delayed care. Indeed, national treatment rates for patients with valve disease and heart failure remain low despite proven therapies and clear indications. Data point to treatment rates of approximately 58% for patients with severe symptomatic aortic stenosis (which is supported by a Class I indication for AVR) and 32% for severe symptomatic mitral regurgitation (which is supported by Class I/IIa indications for surgical or transcatheter repair or replacement).

### SUMMARY

In some aspects, the techniques described herein relate to a method including: determining, by at least one processor, a condition-related criteria associated with diagnosis or treatment or both of a condition; determining, by the at least one processor, a set of condition-related criteria logic rules derived from the condition-related criteria, where the set of condition-related criteria logic rules include logic-based rules that define clinically significant information for the condition; where the set of condition-related criteria logic rules include: at least one health data type associated with the clinically significant information for the condition, and at least one threshold for the at least one health data type; determining, by the at least one processor, at least one clinical data parser configured to parse data associated with the at least one health data type so as to extract the clinically significant information from the data; inputting, by the at least one processor, a plurality of electronic medical record associated with a plurality of patients into the at least one clinical data parser to extract the clinically significant information from each electronic medical record; generating, by the at least one processor, a plurality of normalized patient data objects for the plurality of patients by mapping the clinically significant information extracted from each electronic medical record to a normalized patient data structure; applying, by the at least one processor, the set of condition-related criteria logic rules and the at least one threshold to the plurality of normalized patient data objects to produce, for each normalized patient data object, a clinical relevance indicator indicating a degree of relevance of the clinically significant information of each patient to the condition; and generating, by the at least one processor, a dashboard user interface to display a list of the plurality of patients ordered according to relevance to the condition based at least in part on the degree of relevance of the clinically significant information of each patient to the condition; where the dashboard user interface presents each normalized patient data structure and each electronic medical record associated with each patient.

In some aspects, the techniques described herein relate to a method, where the condition is associated with at least one treatment including at least one of: Left Atrial Appendage Closure (LAAC), pulmonary artery (PA) sensor, mitral transcatheter edge-to-edge repair (TEER), aortic valve replacement (AVR), mitral valve replacement (MVR), implantable cardioverter-defibrillator (ICD), cardiac resynchronization therapy (CRTD), or atrial fibrillation (AF) ablation.

In some aspects, the techniques described herein relate to a method, where the at least one clinical data parser includes at least one severity natural language processor configured to process echocardiogram reports and extract indications of severity of a cardiac condition.

In some aspects, the techniques described herein relate to a method, where the at least one clinical data parser is configured to identify and extract at least one diagnostic code from the plurality of electronic medical records.

In some aspects, the techniques described herein relate to a method, where the at least one clinical data parser is configured to determine at least one admissions statistic associated with at least one patient of the plurality of patients based at least in part on at least one time associated with at least one entry in at least one electronic medical of the plurality of electronic medical records.

In some aspects, the techniques described herein relate to a method, where the at least one clinical data parser is configured to identify and extract at least one laboratory test result; and where the at least one threshold includes at least one threshold value associated with the at least one laboratory test result, the at least one threshold value defining relevance of the at least one laboratory test result to the condition.

In some aspects, the techniques described herein relate to a method, further including: utilizing, by the at least one processor, at least one condition-related criteria machine learning model to ingest the condition-related criteria and, based at least in part on a plurality of trained condition-related criteria machine learning model parameters, output: the at least one health data type associated with the clinically significant information for the condition, and the at least one threshold for the at least one health data type; and generating, by the at least one processor, the set of logic rules based at least in part on: the at least one health data type associated with the clinically significant information for the condition, and the at least one threshold for the at least one health data type.

In some aspects, the techniques described herein relate to a system including: at least one processor which, upon execution of instructions, is configured to: determine a condition-related criteria associated with diagnosis or treatment or both of a condition; determine a set of condition-related criteria logic rules derived from the condition-related criteria, where the set of condition-related criteria logic rules include logic-based rules that define clinically significant information for the condition; where the set of condition-related criteria logic rules include: at least one health data type associated with the clinically significant information for the condition, and at least one threshold for the at least one health data type; determine at least one clinical data parser configured to parse data associated with the at least one health data type so as to extract the clinically significant information from the data; input a plurality of electronic medical record associated with a plurality of patients into the at least one clinical data parser to extract the clinically significant information from each electronic medical record; generate a plurality of normalized patient data objects for the plurality of patients by mapping the clinically significant information extracted from each electronic medical record to a normalized patient data structure; apply the set of condition-related criteria logic rules and the at least one threshold to the plurality of normalized patient data objects to produce, for each normalized patient data object, a clinical relevance indicator indicated a degree of relevance of the clinically significant information of each patient to the condition; and generate a dashboard user interface to display a list of the plurality of patients ordered according to relevance to the condition based at least in part on the degree of relevance of the clinically significant information of each patient to the condition; where the dashboard user interface presents each normalized patient data structure and each electronic medical record associated with each patient.

In some aspects, the techniques described herein relate to a system, where the condition is associated with at least one treatment including at least one of: Left Atrial Appendage Closure (LAAC), pulmonary artery (PA) sensor, mitral transcatheter edge-to-edge repair (TEER), aortic valve replacement (AVR), mitral valve replacement (MVR), implantable cardioverter-defibrillator (ICD), cardiac resynchronization therapy (CRTD), or atrial fibrillation (AF) ablation.

In some aspects, the techniques described herein relate to a system, where the at least one clinical data parser includes at least one severity natural language processor configured to process echocardiogram reports and extract indications of severity of a cardiac condition.

In some aspects, the techniques described herein relate to a system, where the at least one clinical data parser is configured to identify and extract at least one diagnostic code from the plurality of electronic medical records.

In some aspects, the techniques described herein relate to a system, where the at least one clinical data parser is configured to determine at least one admissions statistic associated with at least one patient of the plurality of patients based at least in part on at least one time associated with at least one entry in at least one electronic medical of the plurality of electronic medical records.

In some aspects, the techniques described herein relate to a system, where the at least one clinical data parser is configured to identify and extract at least one laboratory test result; and where the at least one threshold includes at least one threshold value associated with the at least one laboratory test result, the at least one threshold value defining relevance of the at least one laboratory test result to the condition.

In some aspects, the techniques described herein relate to a system, where the at least one processor is further configured to: utilize at least one condition-related criteria machine learning model to ingest the condition-related criteria and, based at least in part on a plurality of trained condition-related criteria machine learning model parameters, output: the at least one health data type associated with the clinically significant information for the condition, and the at least one threshold for the at least one health data type; and generate the set of logic rules based at least in part on: the at least one health data type associated with the clinically significant information for the condition, and the at least one threshold for the at least one health data type.

In some aspects, the techniques described herein relate to a non-transitory computer-readable medium having instructions stored thereon, the instructions being configured to, upon execution, cause at least one processor to perform steps including: obtaining a condition-related criteria associated with diagnosis or treatment or both of a condition; determining a set of condition-related criteria logic rules derived from the condition-related criteria, where the set of condition-related criteria logic rules include logic-based rules that define clinically significant information for the condition; where the set of condition-related criteria logic rules include: at least one health data type associated with the clinically significant information for the condition, and at least one threshold for the at least one health data type; determining at least one clinical data parser configured to parse data associated with the at least one health data type so as to extract the clinically significant information from the data; inputting a plurality of electronic medical record associated with a plurality of patients into the at least one clinical data parser to extract the clinically significant information from each electronic medical record; generating a plurality of normalized patient data objects for the plurality of patients by mapping the clinically significant information extracted from each electronic medical record to a normalized patient data structure; applying the set of condition-related criteria logic rules and the at least one threshold to the plurality of normalized patient data objects to produce, for each normalized patient data object, a clinical relevance indicator indicated a degree of relevance of the clinically significant information of each patient to the condition; and generating a dashboard user interface to display a list of the plurality of patients ordered according to relevance to the condition based at least in part on the degree of relevance of the clinically significant information of each patient to the condition; where the dashboard user interface presents each normalized patient data structure and each electronic medical record associated with each patient.

In some aspects, the techniques described herein relate to a non-transitory computer-readable medium, where the condition is associated with at least one treatment including at least one of: Left Atrial Appendage Closure (LAAC), pulmonary artery (PA) sensor, mitral transcatheter edge-to-edge repair (TEER), aortic valve replacement (AVR), mitral valve replacement (MVR), implantable cardioverter-defibrillator (ICD), cardiac resynchronization therapy (CRTD), or atrial fibrillation (AF) ablation.

In some aspects, the techniques described herein relate to a non-transitory computer-readable medium, where the at least one clinical data parser includes at least one severity natural language processor configured to process echocardiogram reports and extract indications of severity of a cardiac condition.

In some aspects, the techniques described herein relate to a non-transitory computer-readable medium, where the at least one clinical data parser is configured to identify and extract at least one diagnostic code from the at least one diagnostic code from the plurality of electronic medical records.

In some aspects, the techniques described herein relate to a non-transitory computer-readable medium, where the at least one clinical data parser is configured to identify and extract at least one laboratory test result; and where the at least one threshold includes at least one threshold value associated with the at least one laboratory test result, the at least one threshold value defining relevance of the at least one laboratory test result to the condition.

In some aspects, the techniques described herein relate to a non-transitory computer-readable medium, where the instructions further cause at least one processor to perform steps: utilizing at least one condition-related criteria machine learning model to ingest the condition-related criteria and, based at least in part on a plurality of trained condition-related criteria machine learning model parameters, output: the at least one health data type associated with the clinically significant information for the condition, and the at least one threshold for the at least one health data type; and generating the set of logic rules based at least in part on: the at least one health data type associated with the clinically significant information for the condition, and the at least one threshold for the at least one health data type.

### BRIEF DESCRIPTION OF THE DRAWINGS

Various embodiments of the present disclosure can be further explained with reference to the attached drawings, where like structures are referred to by like numerals throughout the several views. The drawings shown are not necessarily to scale, with emphasis instead generally being placed upon illustrating the principles of the present disclosure. Therefore, specific structural and functional details disclosed herein are not to be interpreted as limiting, but merely as a representative basis for teaching one skilled in the art to variously employ one or more illustrative embodiments.

FIG. 1 depicts a system for dynamically analyzing relevance of electronic medical records to reference information in accordance with one or more embodiments of the present disclosure.

FIG. 2 depicts an illustrative set of parsers and a rules engine for dynamically analyzing relevance of electronic medical records to reference information in accordance with one or more embodiments of the present disclosure.

FIG. 3 depicts a block diagram of an exemplary computer-based system and platform for dynamically analyzing relevance of electronic medical records to reference information in accordance with one or more embodiments of the present disclosure.

FIG. 4 depicts a block diagram of another exemplary computer-based system and platform for dynamically analyzing relevance of electronic medical records to reference information in accordance with one or more embodiments of the present disclosure.

FIG. 5 depicts illustrative schematics of an exemplary implementation of the cloud computing/architecture(s) in which embodiments of a system for dynamically analyzing relevance of electronic medical records to reference information may be specifically configured to operate in accordance with some embodiments of the present disclosure.

FIG. 6 depicts illustrative schematics of another exemplary implementation of the cloud computing/architecture(s) in which embodiments of a system for dynamically analyzing relevance of electronic medical records to reference information may be specifically configured to operate in accordance with some embodiments of the present disclosure.

FIGS. 7, 8, 9, 10A, 10B, 10C, 10D, 10E, 11, 12, 13, 14, 15, 16, 17, 18, 19, 20, 21, 22, 23, 24, 25, 26, 27, 28, 29, 30, 31, 32 and 33 depict exemplary screenshots of the graphical user interface having a dashboard and relevant patient lists leveraging the dynamically analyzing relevance of electronic medical records to reference information in accordance with one or more embodiments of the present disclosure.

### DETAILED DESCRIPTION

Various detailed embodiments of the present disclosure, taken in conjunction with the accompanying FIGs., are disclosed herein; however, it is to be understood that the disclosed embodiments are merely illustrative. In addition, each of the examples given in connection with the various embodiments of the present disclosure is intended to be illustrative, and not restrictive.

Throughout the specification, the following terms take the meanings explicitly associated herein, unless the context clearly dictates otherwise. The phrases “in one embodiment” and “According to aspects of one or more embodiments” as used herein do not necessarily refer to the same embodiment(s), though it may. Furthermore, the phrases “in another embodiment” and “in some other embodiments” as used herein do not necessarily refer to a different embodiment, although it may. Thus, as described below, various embodiments may be readily combined, without departing from the scope or spirit of the present disclosure.

In addition, the term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

As used herein, the terms “and” and “or” may be used interchangeably to refer to a set of items in both the conjunctive and disjunctive in order to encompass the full description of combinations and alternatives of the items. By way of example, a set of items may be listed with the disjunctive “or”, or with the conjunction “and.” In either case, the set is to be interpreted as meaning each of the items singularly as alternatives, as well as any combination of the listed items.

As used herein, the terms “clinically relevant” and “clinically significant” and variations thereof refer to a diagnosis, treatment, effect, result, consequence, test, measurement, biomarker or other factor is understood to have a factual effect relative to a particular disease and/or condition. Clinical relevance/significance may be industry accepts, scientifically proven, inferred, or otherwise determined.

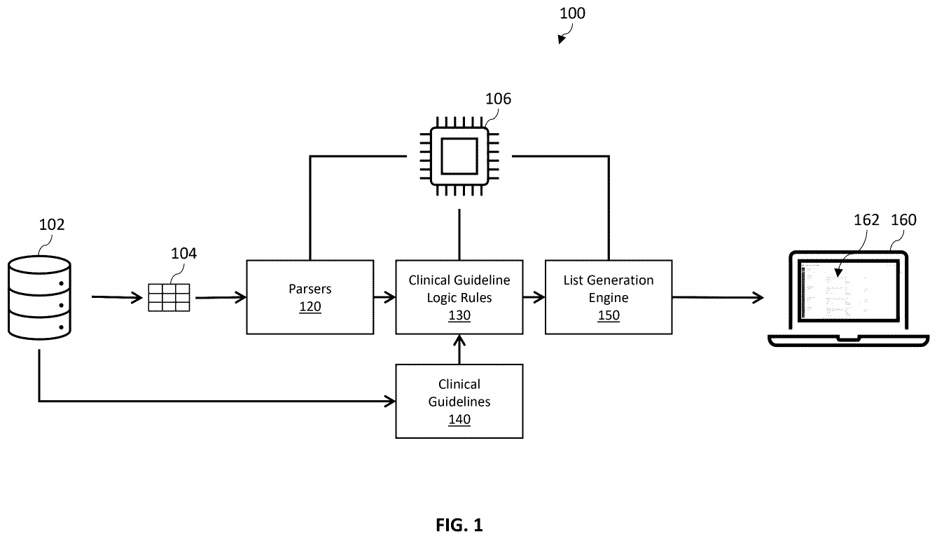

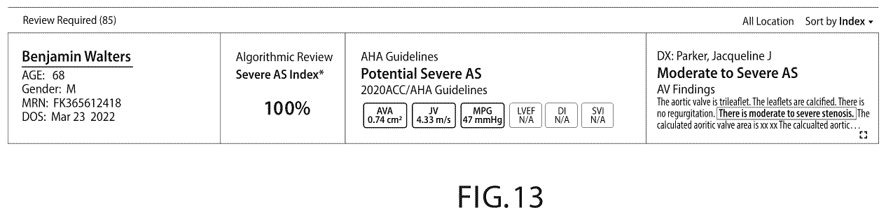

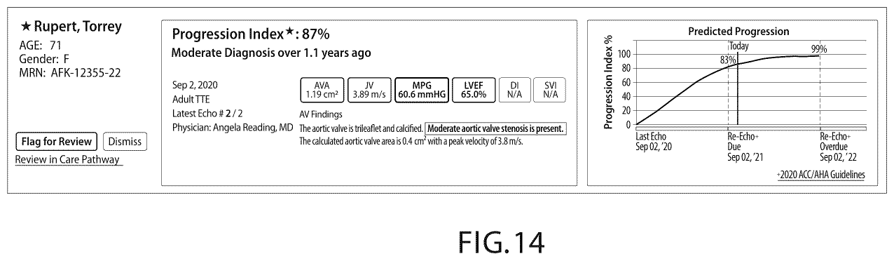

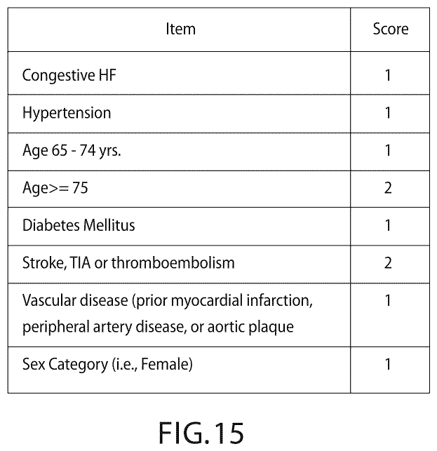

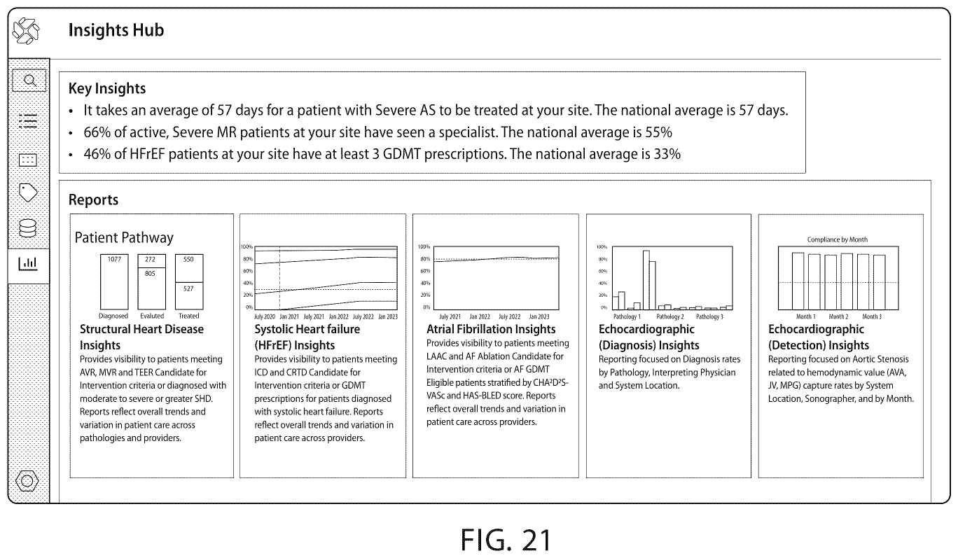

FIGS. 1 through 33 illustrate systems and methods that identify patients who may be indicated for guideline-based therapy (medical or interventional) and warrant a secondary review and, if appropriate, evaluation by a specialist. The following embodiments provide technical solutions and technical improvements that overcome technical problems, drawbacks and/or deficiencies in the technical fields involving electronic medical record processing, analysis and presentation, such as difficulties in an ability to interpret and/or analyze electronic medical records, for example to identify patients meeting particular reference information such as clinical practice guidelines. As explained in more detail, below, technical solutions and technical improvements herein include aspects of improved electronic medical record parsing and analysis that leverages a library of modular parsing tools that are deployed in particular configurations so as to enable a rules engine to score electronic medical records for relevance to particular diseases, conditions and/or treatments. As a result, the technical solutions and improvements herein enable dynamic surfacing of relevant patients as electronic medical records associated with the patients are updated throughout their care. Based on such technical features, further technical benefits become available to users and operators of these systems and methods. Moreover, various practical applications of the disclosed technology are also described, which provide further practical benefits to users and operators that are also new and useful improvements in the art.

Referring to FIG. 1, a system for dynamically analyzing relevance of electronic medical records to reference information is depicted in accordance with one or more embodiments of the present disclosure.

According to aspects of one or more embodiments, a dynamic patient insight platform 100 may obtain electronic medical records 104 for patients of one or more healthcare providers. According to aspects of one or more embodiments, the dynamic patient insight platform 100 may leverage one or more natural language processing (NLP) and/or artificial intelligence (AI) parsers 120, including, according to aspects of one or more embodiments, one or more large language models (LLMs) and/or generative AI models. Using the parsers 120 along with condition-related criteria logic rules 130 derived from condition-related criteria 140, the dynamic patient insight platform 100 may regularly or continuously evaluate the electronic medical records of one or more patients to assess relevance to particular diseases, conditions, treatments, studies, among other healthcare-related information or any combination thereof.

According to aspects of one or more embodiments, the parsers 120 and the condition-related criteria logic rules 130 enable the dynamic patient relevance platform 100 to improve outcomes for patients with cardiovascular disease through earlier identification of disease. Indeed, the dynamic patient relevance platform 100 may enable identification of patients that might be considered for prioritized secondary review to ensure that patients receive guideline-directed care.

According to aspects of one or more embodiments, the parsers 120 and the condition-related criteria logic rules 130 may be implemented in a cloud based, HITRUST compliant platform that aggregates information through a unidirectional connection to a hospital system or site's PACS (Picture Archiving and Communications Systems) system and its EMR database (e.g., Clarity for EPIC customers). Using these tools, the dynamic patient relevance platform 100 may present a patient list GUI 162 on one or more user computing device 160 having one or more modules. For example, the patient list GUI 162 may include Patient List Dashboard displays, for each of several specified cardiovascular disease states, the patients meeting criteria set forth in published clinical practice guidelines (“Guidelines”) consistent with significant disease. According to aspects of one or more embodiments, the Patient Lists may be dynamic, and patients may be included or excluded based upon the status of the most recent list criteria captured. According to aspects of one or more embodiments, the patient list GUI 162 may include database displays that display, e.g., either echo-centric data (e.g., one line per echo or other format or any combination thereof) or patient-centric data (echo report and/or EMR data, e.g., one line per patient or other format or any combination thereof) view where the user can sort or filter with any number of criteria of clinical interest. For example, the patient list GUI 162 may include a visualization of trends at the provider and system level that span valvular heart disease, systolic heart failure, and atrial fibrillation. Such visualizations may track trends including, but not limited to, prescription rates, treatment rates and select echocardiographic data.

According to aspects of one or more embodiments, the electronic medical records 104 may include, without limitation, patient demographics, diagnoses, medical histories, progress notes, vital signs, medical history information, allergies, prescriptions and/or medications, and lab results. The pre-operative data may also include images and/or the associated reports related to the anatomical area of interest. These images may be captured, for example, using Magnetic Resonance Imaging (MRI), Computed Tomography (CT), X-ray, ultrasound, or any other modality known in the art and interpreted in reports created by medical personnel. The pre-operative data may also include quality of life data captured from the patient. For example, in one embodiment, pre-surgery patients use a mobile application (“app”) to answer questionnaires regarding their current quality of life.

According to aspects of one or more embodiments, the electronic medical records 104 or relevant excerpts thereof may be collected and stored in one or more data source(s) 102. According to aspects of one or more embodiments, the data source(s) 102 may include, without limitation, a database, a software-as-a-service or cloud service, a plug-in to an external system, program, application, platform, database, etc., a hard drive, solid-state drive, flash drive, a random-access memory, cache, buffer, or other suitable data source(s) or any combination thereof.

According to aspects of one or more embodiments, the data source(s) 102 may include a combination of manual data entry (e.g., via the computing device 160), databases (e.g., a Clinical Data Warehouse (CDW), PACS, or both) and/or data analytics services, such as one or more databases configured to perform patient- and health-related analytics on electronic medical records. For example, one or more data source(s) 102 may include the PACS, CDW and/or manual entries (e.g., some columns may be populated with manual entries while others may not, such as dismissed information being visible in the database and/or an insights hub, and/or manually entered procedures), where data specifications for data provided to a database and/or to the dynamic patient insight platform may be specified, e.g., as per Table 1 below:

TABLE 1
Data Source Specifications

| Dynamic patient | | |
|---|---|---|
| insight platform | | |
| 100 Module | Data Source | Data Specifications |
| Dynamic patient | PACS + CDW | Relevant cardiovascular CDW information |
| insight | (Clinical Data | pertaining to encounters, medications and |
| platform 100 | Warehouse) + | procedures + and navigator clicks. |
| | Manual entries | Historical data for trends and analysis, |
| | (select) | 3-year CDW + PACS > connection date |
| Database | PACS + | All PACS + CDW echo ONLY if within |
| | CDW + | Disease Progression |
| | Manual | |
| | entries | |
| | (select) | |

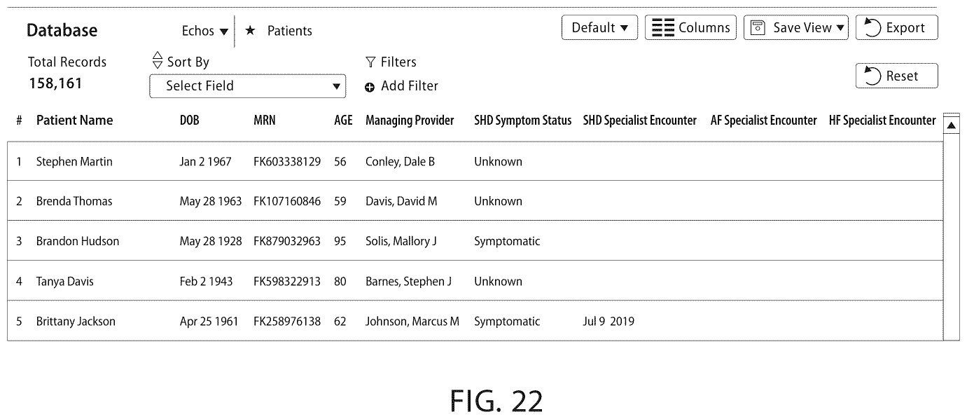

According to aspects of one or more embodiments, the data source(s) 102 may provide users with the flexibility to review various combinations of variables in a list, table or other form (see FIG. 22). According to aspects of one or more embodiments, there may be one or more databases, such as the PACS, the CDW or both, that support the functionality of the dynamic patient insight platform 100 software. According to aspects of one or more embodiments, the data source(s) 102 may reflect the last (latest) date of a diagnosis or procedure or other health-related and/or patient-related data. Users may interact with the data via a patient lists graphical user interface (GUI) 162 on a user computing device 160 in communication with the dynamic patient insight platform 100.

According to aspects of one or more embodiments, in these databases via the patient lists GUI 162, the user can create public and private custom queries, save queries that they want to repeat in the future, add and remove columns, add filters to columns and export a query in a format such as, e.g., in an Excel format, PDF format, CSV format, plain text format, XML format, among others or any combination thereof.

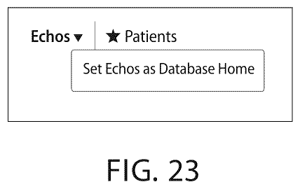

According to aspects of one or more embodiments, using the patient lists GUI 162, users can select either the Echo or Patient Database as a home view by clicking on the expander to the right of the Database name. For example, as illustrated in FIG. 23, a patient database may be currently set as the home view as indicated by the star, and once ‘Set Echocardiograms (“echos”) as Database Home’ is selected from the dropdown arrow, the Echo Database may become the landing page when the database is entered by the user in the future.

According to aspects of one or more embodiments, as detailed below, the parsers 120 may output information extracted from reports in an EMR. According to aspects of one or more embodiments, the parsers 120 may also output interpretations of the EMR including, e.g., the relevance of the patient to a patient- or health-related category based on reference information (e.g., clinical practice guidelines, etc.). According to aspects of one or more embodiments, from the Echo Database, the user may use the patient lists GUI 162 to navigate to a patient's summary of the extracted information and/or interpretations, including a summary of one or more portions of the EMR such as an echo report, by clicking on any given row. From the Patient Database, the user may navigate to a patient's review page by clicking on any given row.

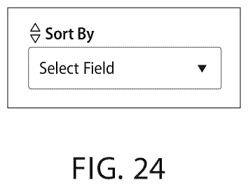

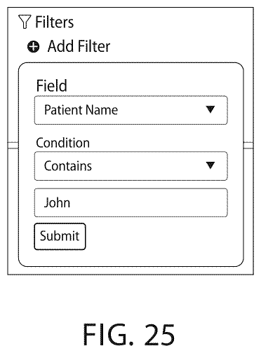

According to aspects of one or more embodiments, lists of patients presented in the patient lists GUI 162 may include one or more filters and/or sorting tools. For example, a ‘Sort By’ element (see FIG. 24) may enable the user to sort in ascending and descending order of a variety of datapoints. In another examples, a Filters/Add Filter element (see FIG. 25) may enable the user to add filters to the database query of patients.

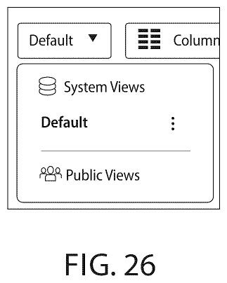

According to aspects of one or more embodiments, the patient lists GUI 162 may include a Field dropdown that is associated with the Database columns that are added to the query. According to aspects of one or more embodiments, the Database Query dropdown may include a System Views element (FIG. 26) created by dynamic patient insight platform 100, a Public View element and/or a Private View element. According to aspects of one or more embodiments, the user may be able to see their own private views, as well as public views created by other users.

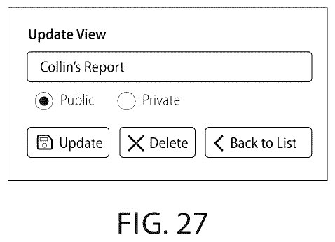

According to aspects of one or more embodiments, the user may interact with the patient lists GUI 162 select a Database query name and open a dialog that allows a user to update the name of a query without changing any characteristics of it, and also enables the user to delete the query (see FIG. 27). According to aspects of one or more embodiments, the patient lists GUI 162 may enable the user to edit or delete their own private and public views but not edit public views created by other dynamic patient insight platform 100 users. Additionally, according to aspects of one or more embodiments, the database may prevent users from creating or editing database query names that already exist across all public and private views created by all dynamic patient insight platform 100 users.

According to aspects of one or more embodiments, a user may save a particular view using, e.g., a Save View button to save updates made to the query, the filters and/or the sorting. According to aspects of one or more embodiments, database queries can be saved as Public or Private. Public queries may be visible to other users. Private queries may be visible only to the user who created and saved it, and/or to other users specified by the user who created it.

According to aspects of one or more embodiments, the patient lists GUI 162 may include a Reset button to remove any filters applied that have not been saved to a current view or a new view. According to aspects of one or more embodiments, the reset button may remove only unsaved filters, or may remove all filters, or any subset of filters.

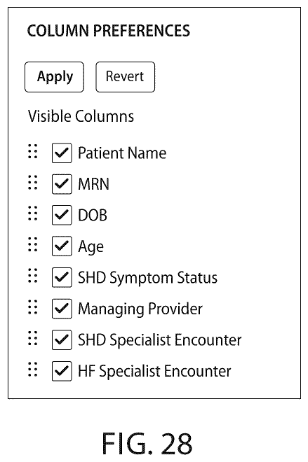

According to aspects of one or more embodiments, the patient lists GUI 162 may include a Column Preferences icon (see FIG. 28) and resulting menu to allow the user to hide or show different columns. According to aspects of one or more embodiments, column ordering may be reorganized by clicking the grid icon (six small squares to the left of the field name) and dragging to the desired location within the list. The selected organization may then be reflected in the display. According to aspects of one or more embodiments, within the Column Preferences menu, the available database columns may be organized within categories to make it easier for users to locate the desired fields for a given query.

According to aspects of one or more embodiments, the patient lists GUI 162 may include an Export button that causes the dynamic patient insight platform 100 to export the list query, e.g., to an Excel spreadsheet or in any other suitable format. The export may include a column per column of the database query.

According to aspects of one or more embodiments, data in the data source(s) 102 may refresh periodically or continuously. According to aspects of one or more embodiments, for periodic refreshes, one or more of the data source(s) 102 may refresh nightly or immediately, depending on what area of the platform the user is interacting with. An example configuration of refresh schedules is presented in Table 2 below, though any refresh schedule may be employed, including a refresh period of, e.g., every 1, 2, 3, 4, 5, 6, 7, 8, 9, 10, 11, 12 or more hours, every night, every day at a specified time, every week, or any other suitable period.

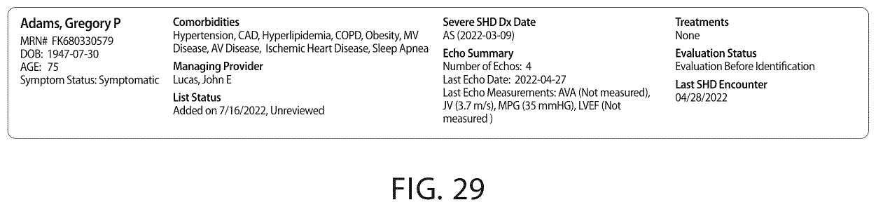

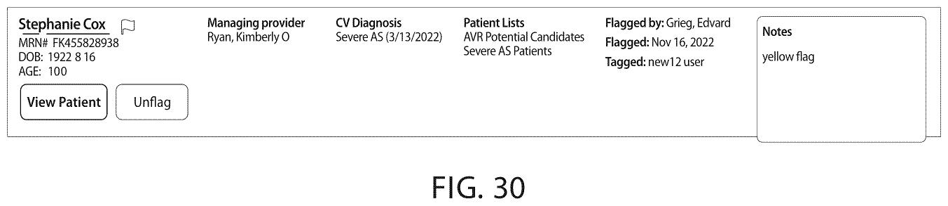

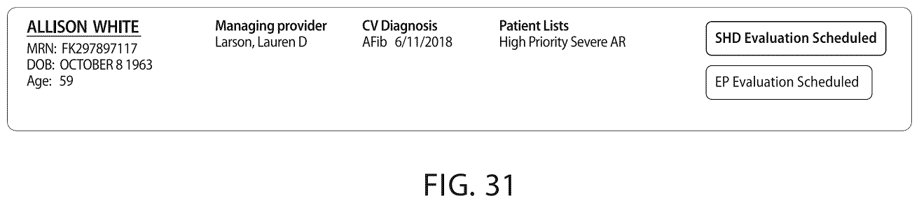

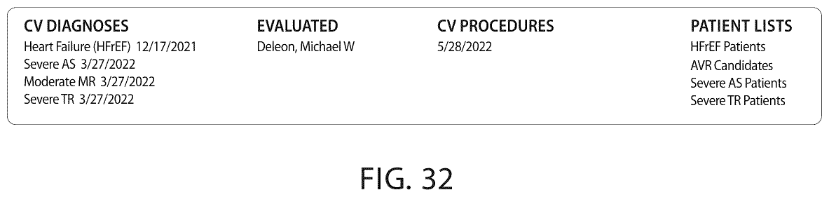

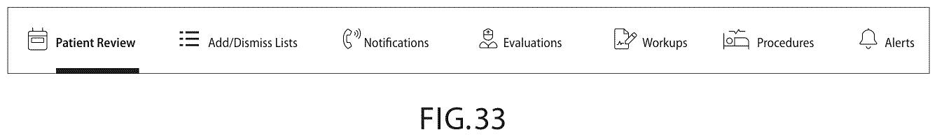

TABLE 2
Example Refresh Schedules for data soure(s) 102.

| Module | Specifics | Date Updates |
|---|---|---|
| Patient List | Dashboard totals, I.e., Severe AS | Immediate |
| Dashboard | (35), HfrEF Patients (10), AVR (55). | |
| Counts | | |
| Patient | Movement of patients on and off | Nightly |
| Lists | lists based on EMR data | |
| Patient | Patients manually added to lists. | Added to |
| Lists | | lists |
| | | Immediately |
| Patient | Patients manually dismissed patients | Removed |
| Lists | | from lists |
| | | Immediately |
| Patient | Information on patient cards on a | Nightly |
| Lists | list (see FIG. 29) | |
| Database | Database column information | Nightly |
| | Note: Patient Labels database | |
| | column will update immediately | |
| Flagged | Patient information on patient | Immediate |
| Items | cards, flags (see FIG. 30) | |
| Module | | |
| Patient | Patient information on patient | Immediate |
| Label | cars, patient labels (see FIG. 31) | |
| Module | Note: CV Diagnoses and Patient | |
| | List information will update nightly. | |
| Patient | Patient Summary: CV Diagnoses, | Nightly |
| Review | Procedure, Evaluations, Patient | |
| page | Lists (see FIG. 32) | |
| | Note: manually added procedures | |
| | update in this section immediately. | |
| | Patients manually Dismissed from | |
| | Lists will reflect in the Patient List | |
| | section immediately. | |
| Patient | Information within Action Bar tabs | Immediate |
| Review | (image), except EMR information | |
| page | in Patient Review event cards | |
| | (see FIG. 33). | |

According to aspects of one or more embodiments, patient lists may be dynamically generated based on the contents of the electronic medical records 104 and the relevance of each patient to a patient or health-related category associated with each list. According to aspects of one or more embodiments, the dynamic patient relevance platform 100 may automatically determine a relevance of a particular patient to a particular category by parsing the data of the electronic medical records 104 to extract clinically relevant data and score the particular patient according to condition-related criteria logic rules 130 derived from condition-related criteria 140.

According to some aspects, the condition-related criteria 140 may include one or more industry guidelines, clinical guidelines, scientific and/or medical publications, among other industry accepted or otherwise authoritative source of criteria for inclusion in a treatment regimen, clinical trial or other action or set of actions. For example, the condition related criteria 140 may include, e.g., clinical guidelines such as one or more American College of Cardiology “ACC,” American Heart Association “AHA,” and/or Heart Failure Society of America “HFSA” clinical practice guidelines or World Health Organization (WHO) and/or National Center for Complementary and Integrative Health (NCCIH) clinical practice guidelines or other clinical practice guidelines for any combination thereof.

According to aspects of one or more embodiments, the parsers 120 may include one or more module parsing tools that can be configured in a combination associated with a particular category. According to aspects of one or more embodiments, the parsers 120 may include machine learning-based NLP to identify and extract semantic concepts. According to aspects of one or more embodiments, the parsers 120 may include machine learning-based NLP to identify and extract key words, key phrases, synonyms and/or other terminology and/or descriptors. According to aspects of one or more embodiments, the parsers 120 may include machine learning-based, similarity measurement-based and/or rules-based keyword searching to identify particular key words, key phrases, synonyms and/or other terminology and/or descriptors. According to aspects of one or more embodiments, the parsers 120 may include machine learning-based, similarity measurement-based and/or rules-based keyword searching to identify particular key-value pairs to extract values for particular data items.

According to aspects of one or more embodiments, the EMR may be in the form of custom reports, such as procedure reports, that may vary based on the source of the EMR, the type of data represented therein, and/or in other ways. According to aspects of one or more embodiments, to ensure that the condition-related criteria logic rules 130 can analyze the data from the parsers 120, the custom reports may be mapped to normalized data fields of the patient lists. According to aspects of one or more embodiments, the normalized data fields may be consistent across all patient lists or may be specific to each particular patient list. According to aspects of one or more embodiments, the custom reports may be mapped to the normalized data fields using one or more pre-defined configurations. Thus, the parsers 120, upon extracting data from an EMR, may structure the data according to the normalized data fields for more efficient and accurate analysis by the condition-related criteria logic rules 130.

According to aspects of one or more embodiments, the electronic medical records 104 may include a plurality of information and/or data types that record clinically significant information of the patient and the patient's health. For example, the information can include doctor provided narratives and/or notes, test results, biometric data, among other information or any combination thereof. For each type of information, e.g., each test, each type of radiology image/scan, each diagnosis or diagnosis type, each type of patient complaint, etc., the parsers 120 may include an associated parser configure to identify and extract the clinically signification information. For example, for radiology reports, a parser may be configured to process the doctor's report and extract descriptors and terminology associated with a particular condition, disease, treatment, etc.

According to aspects of one or more embodiments, the data extracted by the parsers 120 may be analyzed using the condition-related criteria logic rules 130 to determine a relevance indication representing whether or not a particular condition, disease, treatment, etc. is relevant to a particular patient, and/or a degree to which the particular condition, disease, treatment, etc. is relevant to the particular patient. For example, the relevance indication may be a binary classification of “relevant” or “not relevant”, a multiclass classification (e.g., “low relevance,” “medium relevance,” “high relevance” or other set of degrees of relevance), a relevance score quantifying a predicted relevance of the associated patient to a particular condition, disease, treatment, etc. or other relevance indicator or any combination thereof.

According to aspects of one or more embodiments, the condition-related criteria logic rules 130 may include a library of rule configurations, where each rule configuration is associated with a particular condition, disease, treatment, etc. According to aspects of one or more embodiments, each rule configuration of the condition-related criteria logic rules 130 may include one or more logic-based rules that define the clinically significant information for the associated condition, disease, treatment, etc. and how to analyze the clinically significant information. Thus, the condition-related criteria logic rules 130 may also define the parser(s) from the library of parsers 120 to employ to extract inputs from the electronic medical records 104. The rule configuration may thus enable the condition-related criteria logic rules to measure relevance of a patient to the respective condition, disease, treatment, etc. based on the electronic medical records 104 and the output of the parsers 120. For example, the condition-related criteria 140 may indicate a particular treatment for a particular condition based on the presence of certain clinically relevant factors, e.g., based on doctor evaluation, biometric measurements, medical test results, among other factors. Thus, a set of rules may be configured for the particular treatment for the particular condition where the set of rules access the outputs for the parsers that match the clinically-relevant factors, e.g., a parser for parsing a doctors written report in the electronic medical records 104 for relevant descriptors and/or terminology relevant to the particular treatment and/or condition, a parser for extracting measurement values associated with the biometric measurements and/or medical test results in the electronic medical records 104, among other parsers for other factors.

According to aspects of one or more embodiments, the condition-related criteria logic rules 130 may include one or more minimum and/or maximum threshold values for each biometric measurement and/or medical test result associated with an associated condition, disease, treatment, etc. According to aspects of one or more embodiments, the condition-related criteria logic rules 130 may include rules for the data, and thus the parsers, to ingest to create a relevance indicator, and one or more rules specifying an algorithm to combine or otherwise aggregate the data into a final relevance indicator. According to aspects of one or more embodiments, the condition-related criteria logic rules 130 may include one or more metrics quantifying a strength of a relevance of a doctor's written report to an associated condition, disease, treatment, etc. based on a presence one or more relevant descriptors, a frequency of each relevant descriptor that is present, a number of relevant descriptors that are present, a weighting of relevant descriptor, among other quantifications or any combination thereof. According to aspects of one or more embodiments, the condition-related criteria logic rules 130 may aggregate the quantifications of descriptors in the doctor's written report to generate a relevancy score.

According to aspects of one or more embodiments, the condition-related criteria logic rules 130 are based on the condition-related criteria 140. The condition-related criteria 140 may be stored in one or more the data source(s) 102 and accessed to formulate the condition-related criteria logic rules 130. According to aspects of one or more embodiments, the condition-related criteria logic rules 130 may be hand crafted based on expert analysis of the condition-related criteria 140. According to aspects of one or more embodiments, one or more machine learning techniques may be employed, such as machine learning or artificial intelligence (AI) based NLP, to extract and generate logic rules from the condition-related criteria 140.

According to aspects of one or more embodiments, one or more of the parsers 120 and/or the generation of logic rules may be configured to utilize one or more exemplary AI/machine learning techniques chosen from, but not limited to, decision trees, boosting, support-vector machines, neural networks, nearest neighbor algorithms, Naive Bayes, bagging, random forests, large language models (LLMs) and the like. According to aspects of one or more embodiments and, optionally, in combination of any embodiment described above or below, an exemplary neutral network technique may be one of, without limitation, feedforward neural network, radial basis function network, recurrent neural network, convolutional network (e.g., U-net) or other suitable network.

According to aspects of one or more embodiments, an exemplary Large Language Model (LLM) technology may be implemented in one of, but not limited to, the following use-cases: Natural Language Processing (NLP) for querying Electronic Medical Records (EMR), data extraction from clinical notes, patient identification for varied devices and trials through interpretation of unstructured EMR data, or as a companion assistant for guiding users navigating patient records in a Software as a Service (Saas) product. According to aspects of one or more embodiments and, optionally, in combination of any embodiment described above or below, an exemplary implementation of the LLM may be executed as follows:
a. define the LLM architecture, model, and fine-tuning dataset,
b. transfer input data, in the form of unstructured text, to the exemplary LLM model,
c. fine-tune the exemplary model with healthcare-specific data and augment with related technologies,
d. validate and determine the model's accuracy within a predetermined range,
e. apply the exemplary trained model to process and interpret the newly received input data,
f. optionally and in parallel, continue to train the exemplary model with updated or additional data at a predetermined periodicity.

According to aspects of one or more embodiments, a multi-layered approach involving several LLMs may be utilized in tandem with traditional NLP techniques to optimize results. For example, one LLM may be specialized in interpreting general medical terminology while another may focus on extracting pertinent patient information from unstructured data sources. Moreover, the technology may encompass mechanisms for controlling associated computational costs, ensuring efficient and cost-effective operation of the LLM in various implementation scenarios. Furthermore, this LLM technology is applied in a manner that maintains compliance with healthcare data protection regulations, ensuring the confidentiality and integrity of patient information.
a. In additional embodiments and, optionally, in combination of any embodiment described above or below, the exemplary LLM technology may aid in identifying patients as candidates for various devices, treatments, or trials using unstructured data from the EMR by:
b. identifying and extracting relevant patient information from unstructured EMR data,
c. filtering and categorizing the extracted patient data based on guideline directed literature and documentation,
d. employing additional validation mechanisms and technology to further refine identified candidates,
e. securely managing and storing patient data to maintain compliance with healthcare regulations and protect patient privacy.

According to aspects of one or more embodiments and, optionally, in combination of any embodiment described above or below, a companion assistant utilizing LLM technology may aid users of a SaaS product in navigating patient records by:
a. receiving and interpreting natural language queries from users,
b. utilizing trained LLMs to extract and present relevant patient data from an electronic database,
c. employing a user-friendly interface to facilitate intuitive interaction between the user and the system,
d. ensuring the security and privacy of the patient data throughout the interaction by implementing robust cybersecurity protocols.

According to aspects of one or more embodiments, as a result of the condition-related criteria logic rules 130 processing the data extracted by the parsers 120 form the electronic medical records 104, the condition-related criteria logic rules 130 may output a relevance indicator of each patient for each condition, disease, treatment, etc. The relevance indicator indicates a degree of similarity of the information in the electronic medical records 104 of a particular patient to the condition-related criteria 140 of a particular condition, disease, treatment, etc. According to aspects of one or more embodiments, the relevance indicator may be on a scale of 0.0 to 0.1, 0.0 to 10.0, 0 to 10, 1 to 10, 0 to 5, 1 to 5, 0 to 20, 1 to 20, 0 to 25, 1 to 25, 0 to 50, 1 to 50, 0 to 100, 1 to 100 or any other suitable scale. According to aspects of one or more embodiments, the greater the magnitude of the relevance indicator, the greater the degree of relevance. Alternatively, the lesser the magnitude of the relevance indicator, the greater the degree of relevance.

According to aspects of one or more embodiments, relevance indicator of each patient may be determined for each condition, disease, treatment, etc. Thus, the relevance of each patient may be estimated for each condition, disease, treatment, etc. so as to provide a preliminary discovery tool to assist users, such as physicians, in identifying candidates for each condition, disease, treatment, etc.

According to aspects of one or more embodiments, to facilitate user discovery of potentially relevant patients for further assessment, a list generation engine 150 may create lists of patients for each condition, disease, treatment, etc. The lists may include all patients having a relevance indicator meeting a predetermined threshold value, such as a predetermined minimum and/or maximum relevance indicator. According to aspects of one or more embodiments, the lists may include all patients ranked by relevance indicator or may include patients having a relevance indicator in a top N percentile or a top X number of patients.

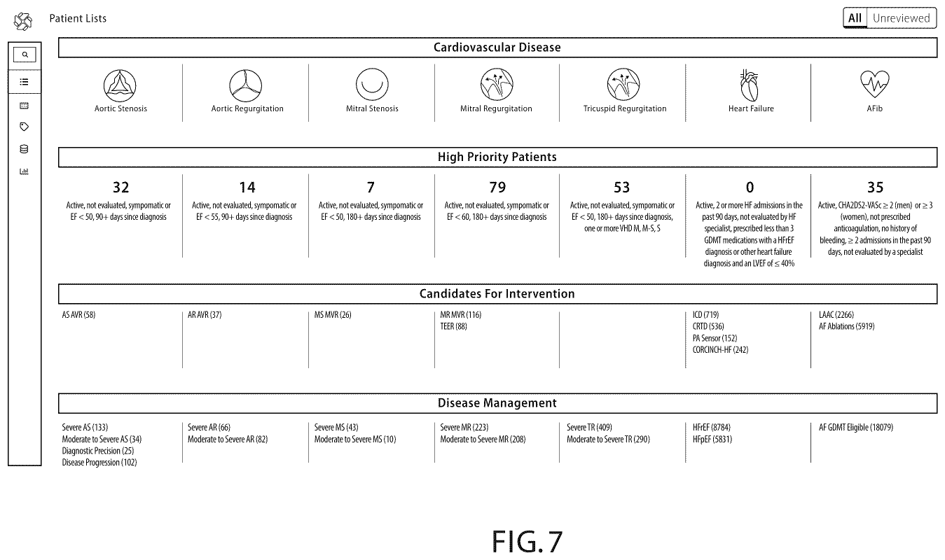

According to aspects of one or more embodiments, the list generation engine 150 may generate a list for each condition, disease, treatment, etc. based on the relevance indicator for each condition, disease, treatment, etc. of each patient. Each list may be cached or otherwise stored in a data storage solution, such as the data source(s) 102 or any other data store or any combination thereof. According to aspects of one or more embodiments, each list may also or instead be output to the patient lists GUI 162 of the user computing device 160. As provided in more detail below with respect to FIGS. 7 through 33, the patient lists GUI 162 may enable .user interaction and exploration of the patient lists and insights thereof.

According to aspects of one or more embodiments, the parsers 120, the condition-related criteria logic rules 130 and/or the list generation engine 150 may be configured to determine relevance associated with one or more diseases, conditions and treatments. For example, based on the condition-related criteria 140 and the condition-related criteria logic rules 130, each cardiac patient may be assessed for the relevance of cardiac treatments based on each cardiac patient's EHR data. As such, the condition-related criteria 140 may set forth criteria for the applicability of each cardiac treatment based on patient health data, tests, metrics or other factors or any combination thereof. Based on the condition-related criteria 140, condition-related criteria logic rules 130 may be produced for configuring one or more of the parsers 120, thresholds, and other post-processing and/or pre-processing to determine the relevance indicator of each cardiac treatment to each cardiac patient. Based on the relevance indicators, the list generation engine 150 may update a patient list for each cardiac treatment with the cardiac patients for which the cardiac treatment is relevant.

According to aspects of one or more embodiments, the cardiac treatments may include, without limitation, Left Atrial Appendage Closure (LAAC), pulmonary artery (PA) sensor, mitral transcatheter edge-to-edge repair (TEER), aortic valve replacement (AVR), mitral valve replacement (MVR), implantable cardioverter-defibrillator (ICD), cardiac resynchronization therapy (CRTD), atrial fibrillation (AF) ablation, among others or any combination thereof, as is be described in more detail below.

According to aspects of one or more embodiments, the parsers 120, the condition-related criteria logic rules 130 and/or the list generation engine 150 may be provided in software instructions on a non-transitory computer readable medium. One or more processors 110 may be in communication with the non-transitory computer readable medium such that the processor 110 may execute the software instructions to implement the parsers 120, the condition-related criteria logic rules 130 and/or the list generation engine 150 and the functions thereof. According to aspects of one or more embodiments, one processor 110 may be used to execute the instructions of all of the parsers 120, the condition-related criteria logic rules 130 and/or the list generation engine 150. According to aspects of one or more embodiments, each of the parsers 120, the condition-related criteria logic rules 130 and/or the list generation engine 150 may have a respective processor 110 or may collective be distributed across multiple processors 110. Any other combination of local, remote, centralized, distributed or other compute paradigm may be employed.

According to aspects of one or more embodiments, the processor 110 may include any type of data processing capacity, such as a hardware logic circuit, for example an application specific integrated circuit (ASIC) and a programmable logic, or such as a computing device, for example, a microcomputer or microcontroller that include a programmable microprocessor. According to aspects of one or more embodiments, the processor 110 may include data-processing capacity provided by the microprocessor. According to aspects of one or more embodiments, the microprocessor may include memory, processing, interface resources, controllers, and counters. According to aspects of one or more embodiments, the microprocessor may also include one or more programs stored in memory.

According to aspects of one or more embodiments, the dynamic patient insight platform 100 may be implemented as cloud service to deliver the patient lists to the user computing device 160 via a network such as the Internet. According to aspects of one or more embodiments, the dynamic patient insight platform 100 may be implemented in a centralized computing system that is local to or remote from the user computing device 160. According to aspects of one or more embodiments, the dynamic patient insight platform 100 may conform to a hybrid architecture having a combination of local and remote processing relative to the user computing device 160.

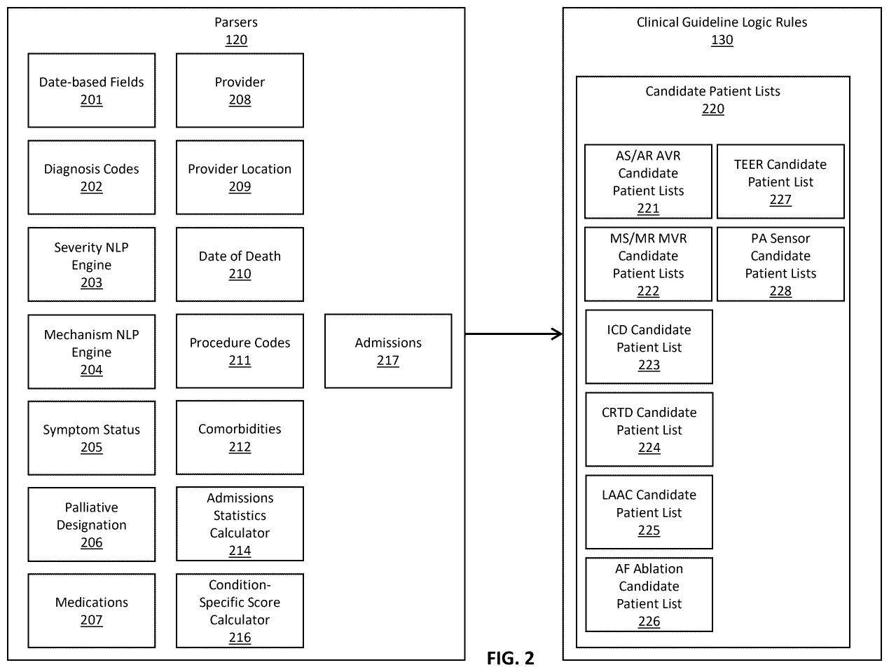

Referring to FIG. 2, a set of parsers and a rules engine for dynamically analyzing relevance of electronic medical records to reference information is depicted in accordance with one or more embodiments of the present disclosure.

According to aspects of one or more embodiments, the parsers 120 may include multiple parsers that each are configured to extract a particular type of clinically relevant data. In an exemplary embodiment illustrated in FIG. 2, the parsers 120 may include, e.g., a date-based fields 201 parser, a diagnosis codes 202 parser, a severity NLP engine 203 parser, a mechanism NLP engine 204 parser, a symptom status 205 parser, a palliative designation 206 parser, a medications 207 parser, a provider 208 parser, a provider location 209 parser, a date of death 210 parser, a procedure code 211 parser, a comorbidities 212 parser, a CHA₂DS₂-VASc Score calculator 214 parser, a HAS-BLED Score calculator 216 parser, an admissions 217 parser, among others or any combination thereof.

According to aspects of one or more embodiments, the date-based fields 201 parser identifies in the electronic medical records 104 the last date of evaluation, diagnosis, procedure, etc. for a patient. In general, the date-based fields 201 parser may be configured to consider all dates to reflect the last date of evaluation, diagnosis, procedure, etc. According to aspects of one or more embodiments, a CV Diagnosis in a Patient Summary section of a Patient Review page of the electronic medical records 104 may show the latest diagnoses for that disease. According to aspects of one or more embodiments, Specialist Evaluations in the Patient Summary section of the Patient Review page of the electronic medical records 104 may show the latest specialist evaluation based on the specialty provider list submitted by the customer. According to aspects of one or more embodiments, CV Procedures in the Patient Summary section of the Patient Review page of the electronic medical records 104 may show all CV Procedures recorded for the patient.

According to aspects of one or more embodiments, the diagnosis codes 202 parser may be configured to identify Diagnosis Codes listed in a patient's electronic medical records 104. Diagnoses for patients may be captured via Diagnosis Codes, except for SHD valve severity which is extracted from echo reports via NLP. Diagnoses written in free text notes within the EMR are not captured or extracted as patient diagnoses in Dynamic patient relevance platform 100.

According to aspects of one or more embodiments, the severity NLP engine 203 parser may employ AI that is trained to read, decipher, and make sense of language. Additional details on the NLP algorithms deployed within dynamic patient relevance platform 100 may include:
a. categorizing based on written severity.
b. not categorizing based on pathology.
c. not categorizing based on measurements.
d. using highest severity found in sentence.

According to aspects of one or more embodiments, the severity NLP engine 203 parser may employ a dictionary of descriptors, such as those of Table 3 below, to identify indications of severity in the electronic medical records 104 based on the training of the AI.

TABLE 3
Dictionary of Descriptors indicative of Severity

| Severity | Definition | Examples not limited to: |
|---|---|---|
| Severe | A diagnosis of Severe was | Severe, Moderately Severe, |
| | extracted from the echo | Critical, Massive, Deluge |
| | report by NLP | |
| Moderate to | A diagnosis of Moderate to | Moderate to severe |
| Severe | Severe was extracted from the | |
| | echo report by NLP | |
| Moderate | A diagnosis of Moderate was | Moderate, |
| | extracted from the echo | “at least moderate” |
| | report by NLP | |
| Mild to | A diagnosis of Mild to | Mild to Moderate |
| Moderate | Moderate was extracted from | |
| | the echo | |
| | report by NLP | |
| Mild | A diagnosis of Mild was | Mild, Trace, Trivial, |
| | extracted from the echo | Physiologic |
| | report by NLP | |
| None or No | The echo report explicitly | No stenosis, |
| Disease | cites the absence of | Without Stenosis, |
| | disease in question | No regurgitation, |
| | or states | Without regurgitation, |
| | explicitly that the | “valve is normal in structure |
| | valve is normal | and function”, “no |
| | | obvious . . . ” |
| Disease of | There is citing of disease, but | “No/without significant |
| Indeterminate | without specification | stenosis/regurgitation” |
| Severity | of severity | “Low gradient AS given the |
| | | severely reduced EF.” |
| | | “Low flow low gradient |
| | | aortic stenosis |
| | | (without stated severity)” |
| | | “No hemodynamically/ |
| | | clinically significant” |
| | | “pseudostenosis” |
| No Diagnosis | No mention of disease | “Valve appears |
| | within echocardiography | grossly normal” |
| | report | |

According to aspects of one or more embodiments, while measurements are not considered in the identification and diagnosis of patients with AS, AR, MS, MR or TR, the measurements may retain a role in Diagnostic Precision, Disease Progression and Echocardiographic Insights (Detection). Notably, potential discrepancies in diagnosis and measurements may appear in the Diagnostic Precision tab and can also be found in the database module.

According to aspects of one or more embodiments, the mechanism NLP engine 204 parser may identify a probable mechanism of a particular disease or condition based on stated descriptors in echocardiographic reports 104. The algorithm may apply rules that were developed in direct collaboration with a panel of external physician experts who informed the sorting mechanisms and qualifying descriptors for each mechanism. The algorithm uses NLP to review the report for the presence of any of the words described in the rules to search for additional required information identified in the rules (such as LVEF), and then to classify the disease or condition according to a set of mechanisms in accordance with the rules.

According to aspects of one or more embodiments, the mechanism NLP engine 204 parser may then stratify the “proxy words” into Tiers 1, 2, or 3 based on the extent to which the “proxy words” are definitively descriptive of a specific mechanism (with Tier 1 words being the most definitive) and identifies additional information (such as LVEF) which, when used in conjunction with the “proxy words” can enhance the accuracy of the classification. According to aspects of one or more embodiments, the mechanism NLP engine 204 parser may assign a classification of one of the mechanisms based on the combinations of proxy words and additional information (i.e., LVEF) in the echocardiographic report,

According to aspects of one or more embodiments, the symptom status 205 parser may identify and extract indications of symptom status. Symptom status may be designated based upon specific disease pathology and labeled as such (SHD Symptomatic, HF Symptomatic, AF Symptomatic). Table 4 below illustrates an example of diagnoses which classify a patient as Symptomatic within the dynamic patient relevance platform 100. If none of the diagnosis codes below are available within a patient's electronic medical records 104, the patient may be designated as “Symptom Status Unknown” for that pathology. Patients may be documented as Symptomatic when two or more disease specific symptoms are documented in the EMR within twelve months prior to diagnosis or any time after diagnosis. The count of two symptoms is based upon two distinct symptoms (e.g., dyspnea and fatigue for heart failure).

TABLE 4
Exemplary Symptom Status Descriptors

| SHD | Heart Failure | Atrial Fibrillation |
|---|---|---|
| Shortness of breath | Shortness of breath | Shortness of breath |
| (dyspnea) | (dyspnea) | (dyspnea) |
| Decreased exercise | Decreased exercise | Decreased exercise |
| tolerance. | tolerance. | tolerance. |
| Heart failure | Syncope or | Angina |
| Angina | presyncope | Chest pain |
| Chest pain | Ascites | Syncope or |
| Syncope or presyncope | Edema | presyncope |
| Ascites | Fatigue/malignant | Fatigue/malignant |
| Edema | fatigue | fatigue |
| Fatigue/malignant | Muscle weakness | Muscle weakness |
| fatigue | (asthenia) | (asthenia) |
| Muscle weakness | Dizziness | Dizziness |
| (asthenia) | Tachycardia | Tachycardia |
| Dizziness | Syncope | Syncope |
| Tachycardia | Palpitations | Palpitations |
| Syncope | Cough | Disorientation |
| Palpitations | Disorientation | Angina |
| Cough | Wheezing | Wheezing |
| Disorientation | Abnormal weight | Unstable angina |
| Angina | gain | |
| Wheezing | | |
| Unstable angina | | |
| Abnormal weight gain | | |

According to aspects of one or more embodiments, the palliative designation 206 parser which may designate patients as palliative or not. Patients may be designated as palliative when:
a. 2+ encounters with palliative care/hospice specialist,
b. 2+ orders for a palliative/hospice evaluation, or
c. Diagnosis Code Z51.5.

According to aspects of one or more embodiments, the palliative designation 206 parser may designate patients as Dismissed where Palliative/Hospice Care has been selected as a dismissal reason by a user within a list for selected disease.

According to aspects of one or more embodiments, the palliative designation 206 parser may not count an order and/or encounter for the same instance (occurring on the same date) as two events.

According to aspects of one or more embodiments, the medications 207 parser may identify and extract medications relative to specific disease states and Patient List criteria based upon prescriptions documented within the EMR, generic and brand name. According to aspects of one or more embodiments, the medications may not be based upon medication lists or encounter notes.

TABLE 5
Exemplary Medications relative to specific disease states
HFrEF Medication Table: ACEi, ARB, ARNi

| ACEi | ACEi | ARB | ARNi |
|---|---|---|---|
| Lotensin | Benazepril | Edarbi | Entresto |
| Capoten | Captopril | Atacand | Sacubitril + |
| | | | Valsartan |
| Vasotec | Enalapril | Teveten | |
| Epaned | Fosinopril | Avapro | |
| Monopril | Lisinopril | Cozaar | |
| Prinivil | Moexipril | Benicar | |
| Zestril | | Micardis | |
| Qbrelis | | Diovan | |
| Univasc | | Azor | |
| Aceon | | Twynsta | |
| Accupril | | Exforge | |
| Altace | | Tribenzor | |
| Mavik | | Edarbyclor | |
| Tarka | | Avalide | |
| Lotrel | | Hyzaar | |
| Prestalia | | Valturna | |
| Vaseretic | | Azilsartan | |
| Prinzide | | medoxomil | |
| Zestoretic | | Candesartan | |
| Uniretic | | Eprosartan | |
| Accuretic | | Irbesartan | |
| Perindopril | | Losartan | |
| Quinapril | | Olmesartan | |
| Ramipril | | Telmisartan | |
| Trandolapril | | Valsartan | |

According to aspects of one or more embodiments, the provider 208 parser may identify a managed by/managing provider designation. According to aspects of one or more embodiments, the provider 208 parser logic may be based upon documented outpatient encounters (clinic, telemedicine, outpatient) within the EMR data available. For encounters where the “visit provider” is invalid or blank, the encounter “referring provider” may be utilized for Managing Provider designation logic. Cardiovascular (CV) encounters are prioritized over PCP encounters for assignation utilizing the following logic:
a. Outpatient encounters out to five years, or the duration of the retrospective data provided, are examined.
b. If there is more than one CV provider, the CV provider with the most encounters is designated as the managing provider.
c. If there are no CV encounters, the PCP (Primary Care, General Medicine, Internal Medicine) with the most encounters is designated as the managing provider.
d. If multiple providers have the same number of encounters, the provider seen most recently is selected.
e. If there are no outpatient encounters, no managing provider is assigned.

According to aspects of one or more embodiments, the provider location 209 parser may determine a managing provider's location. Managing Provider Location may be based upon the Managing Provider's practice as listed within a Provider Roster.

According to aspects of one or more embodiments, the date of death 210 parser may identify and extract a date of death (where applicable). According to aspects of one or more embodiments, the date of death reflects the date documented within CDW extract. When a patient status is expired within the CDW, but no date of death is available the date of last patient record update may be substituted. If a patient is manually Dismissed: Expired the date of entry may be captured as Date of Death.

According to aspects of one or more embodiments, the procedure code 211 parser may determine a procedure relation to a “treated” patient state in the electronic medical records 104. SHD patients are considered ‘Treated’ only in relation to the valve disease under review.
a. Aortic Stenosis or Regurgitation:
   i. surgical and transcatheter aortic valve replacement, including Bentall procedure or heart transplant.
b. Mitral Stenosis or Regurgitation:
   i. surgical mitral valve (MV) replacement or repair,
   ii. transcatheter MV replacement or repair, including transcatheter edge-to-edge repair (TEER) or heart transplant.
c. Tricuspid Regurgitation:
   i. surgical and transcatheter tricuspid valve replacement or repair or heart transplant
d. LAAC (surgical and transcatheter):
   i. Surgical or transcatheter LAAC procedure
   ii. excludes a patient from the AF High Priority Patient List as Treated, but does not prohibit them from inclusion within the AF GDMT Eligible or other AF related Patient Lists

According to aspects of one or more embodiments, dilation, release, valvuloplasty, valvotomy may be not considered ‘Treated’ procedures within the dynamic patient relevance platform 100 data sets with one exception, 33460 “Valvectomy, tricuspid valve, with cardiopulmonary bypass” for treatment of the tricuspid valve.

According to aspects of one or more embodiments, a Heart Transplant may be considered a treatment for all dynamic patient relevance platform 100 pathologies.

According to aspects of one or more embodiments, the procedure code 211 parser may identify all procedures as valid whether manually entered or extracted from EMR via procedure codes. Examples of procedure relationships to “treated” patients are provided in Table 6 below:

TABLE 6
Example Procedure relationships to “Treated” Patients

| | Pathology/Patient List |
|---|---|
| Procedure | “Treated” |
| SAV Repair | N/A |
| SAV Replacement | Aortic Valve |
| TAVR | Aortic Valve |
| BAV | |
| SMV Repair | Mitral Valve |
| SMV Replacement | Mitral Valve |
| TMVr/TEER | Mitral Valve |
| TMVR | Mitral Valve |
| STV Repair | Tricuspid Valve |
| STV Replacement | Tricuspid Valve |
| TTV Repair | Tricuspid Valve |
| TTV Replacement | Tricuspid Valve |
| Heart Transplant | AV/MV/TV/HFrEF/AF |
| LAAC | AF High Priority |
| (Surgical/Transcatheter) | |

According to aspects of one or more embodiments, the comorbidities 212 parser may designate a patient's comorbidities based on the electronic medical records 104 specify one or more of a set of diseases and/or conditions. For example, the comorbidities 212 parser may be configured to identify in the electronic medical records:
a. Atrial Fibrillation (AF)
b. Anemia
c. AV Disease (defined as moderate or greater diagnosis of Aortic Valve)
d. Coronary Artery Disease (CAD)
e. Metastatic Cancer
f. Cerebrovascular Disease
g. Conduction Delay
h. COPD
i. Dementia
j. Diabetes
k. End Stage Renal Disease
l. Heart Failure (HF)
m. Risk of Bleed
n. Hyperlipidemia
o. Hypertension
p. Hypotension
q-Ischemic Heart Disease
r. Liver Disease
s. MV Disease (defined as moderate or greater diagnosis of Mitral Valve)
t. Obesity
u. Peripheral Vascular Disease
v. Renal failure or kidney disease
w. Sleep Apnea
x. Syncope
y. TV Disease (defined as moderate or greater diagnosis of Tricuspid Valve)

According to aspects of one or more embodiments, the CHA₂DS₂-VASc Score calculator 214 parser may extract one or more factors associated with CHA₂DS₂-VASc to formulate a score. According to aspects of one or more embodiments, the criteria of the CHA₂DS₂-VASc Score, as defined in the 2019 AHA/ACC/HRS Focused Update of the 2014 AHA/ACC/HRS Guideline for the Management of Patients with Atrial Fibrillation, are calculated based upon extracted diagnosis codes for the following:

TABLE 7
Exemplary Criteria for CHA₂DS₂-VASc

| Item | Score |
|---|---|
| Congestive HF | 1 |
| Hypertension | 1 |
| Age 65-74 years | 1 |
| Age ≥65 | 2 |
| Diabetes Mellitus | 1 |
| Stroke, TIA or | 2 |
| thromboembolism | |
| Vascular disease (prior | 1 |
| myocardial infarction, | |
| peripheral artery disease or | |
| aortic plaque) | |
| Sex Category (I.e., female) | 1 |

According to aspects of one or more embodiments, the HAS-BLED Score calculator 216 parser may extract one or more factors associated with HAS-BLED to formulate a score. According to aspects of one or more embodiments, the criteria of the HAS-BLED Score may be calculated with diagnoses that are extracted via codes. The codes that the dynamic patient relevance platform 100 uses for those respective criteria are as follows:

TABLE 8
Exemplary Criteria for HAS-BLED Score

| Item | Score |
|---|---|
| Hypertension | 1 |
| Abnormal Renal/Liver | ½ |
| Function | |
| Stroke | 1 |
| Bleeding Tendency or | 1 |
| Predisposition | |
| Labile INR | 1 |
| Elderly (e.g., Age >65 years) | 1 |
| Current Drugs (e.g., | ½ |
| Concomitant Aspirin, or | |
| NSAIDS) or Alcohol Use | |

According to aspects of one or more embodiments, the admissions 217 parser may be configured to aggregate statistics associated with a patient's admissions to one or more healthcare institutions. For example, the admissions 217 parser may recognize and extract an admission that lasts, e.g., for greater than 24 hours. Admissions with a discharge less than 24 hours are not included within the Total Admissions numbers. Disease specific admissions (HF, AF, Bleed Related) may be based upon a disease diagnosis code captured within the EMR diagnosis table or admission/discharge associated EMR problem list.

According to aspects of one or more embodiments, the condition-related criteria logic rules 130 may obtain the data output by the parsers 120 to determine relevance indicators for each of a set of candidate patient lists 220, including rule configurations for leveraging the data output by the parsers to generate candidate patient lists.

### Examples of Patient Lists for Illustrative Cardiac Treatments

According to aspects of one or more embodiments, the candidate patient lists 220 may include AS and/or AR (AS/AR) AVR candidate patients list 221. The AS/AVR Candidate patient lists 221 may be populated with patients that meet Class I indications for intervention according to the 2020 ACC/AHA Guidelines for the Management of Patients with Valvular Heart Disease. Where valve hemodynamics are criteria for inclusion or exclusion, data is based upon the most recent echocardiogram. Patients previously receiving a heart transplant and/or an aortic valve replacement are excluded from the AVR Candidate Patient Lists.

According to aspects of one or more embodiments, patients meeting any of the following criteria are placed on the AS/AVR Candidate patient list 221:
a. Severe symptomatic AS
b. Severe AS, SHD symptoms unknown, EF<50%
c. Low flow low gradient severe AS, symptomatic (EF<50% AND JV<4 AND AVA≤1.0)
d. Low flow low gradient severe AS, symptomatic (EF≥50% AND JV<4 AND AVA≤1.0)

According to aspects of one or more embodiments, patients meeting any of the following criteria may be excluded from the AR/AVR Candidate patient list 221:
a. Severe, SHD symptomatic AR
b. Severe AR, SHD symptoms unknown, EF≤55%

According to aspects of one or more embodiments, visible within the Patient List are select Insights. These Insights may reflect data based upon the current Patient List selected within the AS or AR AVR Candidate patient list filtering and/or sorting mechanism (e.g., a dropdown (for example with selectable options for System Views: Candidate/Dismissed or Public/Private saved queries)) or any currently applied manual filters.
a. AS AVR Class I Indications subset rate and count
b. AR AVR Class I Indications subset rate and count
c. SHD Specialist Evaluation rate and count by Managing Provider and Overall

According to aspects of one or more embodiments, the candidate patient lists 220 may include MS and/or MR (MS/MR) MVR Candidate Patient Lists 222. The MS/MR MVR Candidate patient lists 222 may be populated with patients that meet Class I indications for intervention, consistent with the 2020 ACC/AHA Guidelines for the Management of Patients with Valvular Heart Disease. Where valve hemodynamics are criteria for inclusion or exclusion data is based upon the most recent echocardiogram. Patients previously receiving a heart transplant and/or a mitral valve replacement or repair are excluded from the MVR Candidate Patient Lists.

According to aspects of one or more embodiments, patients meeting any of the following criteria are placed on the MS/MR MVR Candidate patient list 221:
a. Severe, degenerative or mixed, SHD symptomatic MR
b. Severe MR, primary or mixed, SHD symptoms unknown, EF≤60% OR LVESD≥40%

According to aspects of one or more embodiments, patients meeting any of the following criteria may be excluded from the MS/MR MVR Candidate patient list 221:
a. Severe, SHD symptomatic MS

According to aspects of one or more embodiments, visible within the Patient List may be select Insights. These Insights may reflect data based upon the current Patient List selected within the MS and/or MR MVR Candidate patient list 222 filtering and/or sorting mechanism (e.g., a dropdown (for example with selectable options for System Views: Candidate/Dismissed or Public/Private saved queries)) or any currently applied manual filters.
a. MS MVR Class I Indication subset
b. MR MVR Class I Indications subset rate and count
c. SHD Specialist Evaluation rate and counts by Managing Provider and Overall

According to aspects of one or more embodiments, patients meeting any of the following criteria may be placed on the TEER Candidate patient list 227:
a. Severe MR, degenerative, symptomatic HF
b. Severe MR, secondary, symptomatic HF, ejection fraction (EF) of 20-50% or LVESD<−70 mm (or blank) and PASP<=70 (or blank)

According to aspects of one or more embodiments, patients meeting any of the following criteria may be excluded from the TEER Candidate patient list 227:
a. Prior surgical mitral valve repair or replacement (SMVR/r), TMVR, TMVr, LVAD or Heart Transplant
b. Adverse effects of anticoagulant
c. Dermatitis due to metal
d. Endocarditis <30 days
e. Severe Dementia

Accordingly, according to aspects of one or more embodiments, the parsers 120 may extract data from an EMR, such as with the severity NLP engine 203, diagnosis codes 202, procedure codes 211, comorbidities 212, condition-specific score calculator 216, among others, to determine whether the EMR indicates any of the inclusion and/or exclusion criteria listed above. Thus, by deploying one or more of the parsers 120 to extract particular information from written and quantitative information in an EMR, the EMR may be assessed against reference information include clinical guidelines such as the criteria above.

According to aspects of one or more embodiments, visible within the Patient List may be select Insights-generated by the parsers 120 and/or additional data analysis algorithms. These Insights may reflect data based upon the current Patient List selected within the TEER Candidate patient list 227 filtering and/or sorting mechanism (e.g., a dropdown (for example with selectable options for System Views: Candidate/Dismissed or Public/Private saved queries)) or any currently applied manual filters.
a. TEER Indication subset
b. TEER Indications subset rate and count
c. SHD Specialist Evaluation rate and counts by Managing Provider and Overall

According to aspects of one or more embodiments, the candidate patient lists 220 may include ICD Candidate Patient List 223. According to aspects of one or more embodiments, patients on the ICD Candidate patient list 223 meet criteria outlined in the ‘2017 AHA/ACC/HRS Guideline for Management of Patients with Ventricular Arrhythmias and the Prevention of Sudden Cardiac Death’ and the Class I Indications. Where LVEF is required criteria for inclusion of a patient within the ICD Candidate patient list, and the L VEF is blank in the most recent echo report, the last known LVEF captured within historical echo reports may be utilized for Candidate patient list inclusion.

According to aspects of one or more embodiments, patients meeting any of the following criteria are placed on the ICD Candidate Patient List 223:
a. LVEF≤35% AND nonischemic cardiomyopathy, HF symptomatic
b. LVEF≤35% AND Ischemic Heart Disease OR MI and HF symptomatic
c. LVEF≤30% AND Ischemic Heart Disease OR MI

According to aspects of one or more embodiments, patients meeting any of the following criteria may be excluded from the ICD Candidate Patient List 223:
a. Prior MI within 40 days
b. Prior PCI or CABG within 90 days
c. Prior ICD or CTRD procedure
d. DNR Directive (z66) coded within the EMR.
e. Metastatic cancer diagnosis coded within the EMR.

According to aspects of one or more embodiments, visible within the Patient List are select Insights. These Insights may reflect data based upon the current Patient List selected within the ICD Candidate patient list 223 filtering and/or sorting mechanism (e.g., a dropdown (for example with selectable options for System Views: Candidate/Dismissed or Public/Private saved queries)) or any currently applied manual filters.
a. ICD Class I Indications subset rate and count
b. HF and EP Specialist Evaluation rate and counts by Managing Provider and Overall
c. HFrEF GDMT and Diuretic Prescription count by Medication Type

According to aspects of one or more embodiments, the candidate patient lists 220 may include CRTD Candidate Patient List 224. Patients on the CRTD Candidate patient list 224 meet either Class I or Class Ila criteria for intervention based on the 2022 AHA/ACC/HFSA Heart Failure Guideline for the Management of Heart Failure. Where LVEF is required criteria for inclusion of a patient within the CRTD Candidate patient list, and the LVEF is blank in the most recent echo report, the last known LVEF captured within historical echo reports may be utilized for Candidate patient list inclusion.

According to aspects of one or more embodiments, patients meeting any of the following criteria are placed on the CRTD Candidate Patient List 224:
a. LVEF≤35% AND HF symptomatic AND LBBB
b. LVEF≤35% AND HF symptomatic AND RBBB OR Intraventricular Conduction Delay (BBB)

According to aspects of one or more embodiments, patients meeting any of the following criteria may be excluded from the CRTD Candidate Patient List 224:
a. Patient has no recorded HF symptoms.
b. Prior CTRD procedure
c. DNR Directive (z66) coded within the EMR.
d. Metastatic cancer diagnosis coded within the EMR.

According to aspects of one or more embodiments, visible within the Patient List may be select Insights. These Insights may reflect data based upon the current Patient List selected within the CRTD Candidate patient list 224 filtering and/or sorting mechanism (e.g., a dropdown (for example with selectable options for System Views: Candidate/Dismissed or Public/Private saved queries)) or any currently applied manual filters.
a. CRTD Class I and IIa Indications subset rate and count
b. HF and EP Specialist Evaluation rate and counts by Managing Provider and Overall
c. HFrEF GDMT and Diuretic Prescription count by Medication Type

According to aspects of one or more embodiments, patients meeting any of the following criteria are placed on the pulmonary artery (PA) Sensor Candidate Patient List 228 associated with implanting a PA sensor:
a. Symptomatic HF, >=2 admissions within 12 months, diuretic prescription within 12 months

According to aspects of one or more embodiments, patients meeting any of the following criteria may be excluded from the PA Sensor Candidate List 228:
a. Prior PA Sensor implant.

According to aspects of one or more embodiments, visible within the Patient List may be select Insights. These Insights may reflect data based upon the current Patient List selected within the PA Sensor Candidate patient list 228 filtering and/or sorting mechanism (e.g., a dropdown (for example with selectable options for System Views: Candidate/Dismissed or Public/Private saved queries)) or any currently applied manual filters.
a. HF Evaluation rate and counts by Managing Provider and Overall
b. GDMT Prescription count by Medication Type
c. GDMT Prescriptions by Managing Provider

According to aspects of one or more embodiments, the candidate patient lists 220 may include LAAC Candidate Patient List 225. Patients on the LAAC (Left Atrial Appendage Closure) Candidate patient list meet the following criteria. These criteria are based on the ‘2019 AHA/ACC/HRS Focused Update of the 2014 AHA/ACC/HRS Guideline for the Management of Patients with Atrial Fibrillation,’ the ‘2023 SCAI/HRS Expert Consensus Statement on Transcatheter Left Atrial Appendage Closure’ and relevant commercial product labeling and patient identification tools.

According to aspects of one or more embodiments, patients meeting any of the following criteria are placed on the LAAC Candidate Patient List 225 for the base Population where:
a. Atrial Fibrillation diagnosis (e.g., within that last 24 months)
b. CHA₂DS₂-VASc score≥2 (male) or ≥3 (female)
c. HAS-Bled score≥3

According to aspects of one or more embodiments, patients meeting any of the following criteria may be excluded from base population for the LAAC Candidate Patient List 225:
a. Previous LAAC procedure (surgical or transcatheter)
b. Moderate or greater mitral stenosis
c. Known allergy to nitinol diagnosis coded within the EMR.
d. Metastatic cancer diagnosis coded within the EMR.

According to aspects of one or more embodiments, patients meeting any of the following criteria may be excluded from LAAC Candidate Patient List 225, but included within the Base Population list where:
a. Patients with a prosthetic valve (as indicated by ICD/CPT procedure codes or NLP of the most recent echocardiogram)
b. Patients who have not been prescribed an anticoagulant.
c. Patients without a diagnosed history of or risk of falls/bleed or a “risk” of bleed/fall related diagnosis code within the last 24 months, but not limited to after the anticoagulation prescription.

According to aspects of one or more embodiments, the LAAC Candidate patient list 225 may include a “LAAC Candidate Criteria: High Priority” filter where:
a. Age≥60
b. CHA₂DS₂-VASc score≥5
c. HAS-BLED score≥4
d. ≥2 Bleeding related admission within a 12 month period.

References to anticoagulant within Dynamic patient relevance platform 100 are specific to documentation and extraction of prescriptions within the EMR dataset and are not based upon consult notes or “med lists.”

According to aspects of one or more embodiments, fall/risk of bleed consists of patients with one or more of the following diagnosis codes within the EMR.

TABLE 9
Example Designated LAAC Related Codes

| Brain, GI or | | Diagnosis |
|---|---|---|
| Risk | | ICD- |
| Designation | Diagnosis | 10 Code |
| Risk | Adverse Effect of Anticoagulants | T45.515 |
| Brain | Cerebral or Intracranial Bleeding | D18.02 |
| Risk | Thrombocytopenia | D69.6 |
| Brain | Nontraumatic Subarachnoid Hemorrhage | I60 |
| Brain | Nontraumatic Intracerebral Hemorrhage | I61 |
| Brain | Unspecified Nontraumatic Intracranial | I62 |
| | Hemorrhage | |
| GI | Esophageal Ulcer with Bleeding | K22.11 |
| GI | Acute gastric ulcer with hemorrhage | K25.0 |
| GI | Acute gastric ulcer with both hemorrhage | K25.2 |
| | and perforation | |
| GI | Chronic or unspecified gastric ulcer with | K25.4 |
| | hemorrhage | |
| GI | Chronic or unspecified gastric ulcer with both | K25.6 |
| | hemorrhage and perforation | |
| GI | Acute duodenal ulcer with hemorrhage | K26.0 |
| GI | Acute duodenal ulcer with both hemorrhage | K26.2 |
| | and perforation | |
| GI | Chronic or unspecified duodenal ulcer | K26.4 |
| | with hemorrhage | |
| GI | Chronic or unspecified duodenal ulcer with both | K26.6 |
| | hemorrhage and perforation | |
| GI | Acute peptic ulcer, site unspecified, | K27.0 |
| | with hemorrhage | |
| GI | Acute peptic ulcer, site unspecified, with both | K27.2 |
| | hemorrhage and perforation | |
| GI | Chronic or unspecified peptic ulcer, site | K27.4 |
| | unspecified, with hemorrhage | |
| GI | Chronic or unspecified peptic ulcer, | K27.6 |
| | site unspecified, | |
| | with both hemorrhage and perforation | |
| GI | Acute gastrojejunal ulcer with hemorrhage | K28.0 |
| GI | Acute gastrojejunal ulcer with both | K28.2 |
| | hemorrhage and perforation | |
| GI | Chronic or unspecified gastrojejunal ulcer | K28.4 |
| | with hemorrhage | |
| GI | Chronic or unspecified gastrojejunal ulcer with | K28.6 |
| | both hemorrhage and perforation | |
| GI | Acute gastritis with bleeding | K29.01 |
| GI | Chronic superficial gastritis with bleeding | K29.31 |
| GI | Chronic atrophic gastritis with bleeding | K29.41 |
| GI | Unspecified chronic gastritis with bleeding | K29.51 |
| GI | Other gastritis with bleeding | K29.61 |
| GI | Gastritis, unspecified with bleeding | K29.71 |
| GI | Duodenitis with bleeding | K29.81 |
| GI | Gastroduodenitis, unspecified with bleeding | K29.91 |
| GI | Diverticular disease of intestine with bleeding | K57.01 |
| GI | Diverticulosis of small intestine without | K57.11 |
| | perforation or abscess with bleeding | |
| GI | Diverticulitis of small intestine without | K57.13 |
| | perforation or abscess with bleeding | |
| GI | Diverticulitis of large intestine with | K57.21 |
| | perforation and abscess with bleeding | |
| GI | Diverticulosis of large intestine without | K57.31 |
| | perforation or abscess with bleeding | |
| GI | Diverticulitis of large intestine without | K57.33 |
| | perforation or abscess with bleeding | |
| GI | Diverticulitis of both small and large intestine | K57.41 |
| | with perforation and abscess with bleeding | |
| GI | Diverticulosis of both small and large intestine | K57.51 |
| | without perforation or abscess with bleeding | |
| GI | Diverticulitis of both small and large intestine | K57.53 |
| | without perforation or abscess with bleeding | |
| GI | Diverticulitis of intestine, part unspecified, | K57.81 |
| | with perforation and abscess | |
| GI | Diverticulosis of intestine, part unspecified, | K57.91 |
| | without perforation or abscess with bleeding | |
| GI | Diverticulitis of intestine, part unspecified, | K57.93 |
| | without perforation or abscess with bleeding | |
| GI | Hemorrhage of anus and rectum | K62.5 |
| GI | Hemoperitoneum (ntra-abdominal hemorrhage | K66.1 |
| | or intraperitoneal hemorrhage) | |
| | Hematemesis (vomiting of blood) | K92.0 |
| GI | Gastrointestinal hemorrhage, unspecified | K92.2 |
| | Hemarthrosis | M25.00 |
| | Hemarthrosis | M25.019 |
| | Hemarthrosis | M25.029 |
| | Hemarthrosis | M25.039 |
| | Hemarthrosis | M25.049 |
| | Hemarthrosis | M25.059 |
| | Hemarthrosis | M25.069 |
| | Hemarthrosis | M25.073 |
| | Hemarthrosis | M25.076 |
| | Hemarthrosis | M25.08 |
| | Nontraumatic hematoma of soft tissue | M79.81 |
| Risk | Repeated falls | R29.6 |
| Risk | History Of Falling | Z91.81 |

According to aspects of one or more embodiments, visible within the Patient List may be select Insights. These Insights may reflect data based upon the current Patient List selected within the LAAC Candidate patient list filtering and/or sorting mechanism (e.g., a dropdown (for example with selectable options for System Views: Candidate/Dismissed or Public/Private saved queries)) or any currently applied manual filters.
a. LAAC Specialist Evaluation rate and count by Managing Provider and Overall
b. CHA₂DS₂-VASc and HAS-BLED score ranges

According to aspects of one or more embodiments, the candidate patient lists 220 may include AF Ablation Candidate Patient List 226. Patients on the AF Ablations Candidate patient list 226 meet the following criteria. These criteria are based on the Joglar J A, Chung M K, Armbruster A L, et al. 2023 ACC/AHA/ACCP/HRS Guideline for the Diagnosis and Management of Atrial Fibrillation: A Report of the American College of Cardiology/American Heart Association Joint Committee on Clinical Practice Guidelines [published correction appears in Circulation. 2024 Jan. 2; 149 (1):e167]. Circulation. 2024; 149 (1):e1-e156. doi:10.1161/CIR.0000000000001193

According to aspects of one or more embodiments, patients meeting the following criteria may be included in the AF Ablation Candidate patient list 226:
a. limited to within the last 24 months
b. Symptomatic AF (not paroxysmal), AAD prescribed:
   i. Antiarrhythmic (AAD) for AF Rhythm Control per guidelines is limited to: Dofetilide, Dronedarone, Flecainide, Propafenone, Amiodarone, Sotalol,
c. Symptomatic AF (not paroxysmal), no AAD prescribed
d. Paroxysmal Symptomatic AF, AAD prescribed
e. Paroxysmal Symptomatic AF, no AAD prescribed

“AF (not paroxysmal) and HFrEF, prescribed 4 HFrEF GDMT, unknown AF symptomsAccording to aspects of one or more embodiments, patients meeting the following criteria may be excluded from the AF Ablation Candidate patient list 226:
a. Previous AF Ablation, or
b. A last AF diagnosis greater than 24 months.

According to aspects of one or more embodiments, the dynamic patient relevance platform 100 may define an ablation procedure as an “AF Ablation” when the patient undergoes a surgical, and/or transcatheter ablation and has a recorded AF diagnosis code within the EMR.

According to aspects of one or more embodiments, visible within the Patient List may be select Insights. These Insights may reflect data based upon the current Patient List selected within the AF Ablation Candidate patient list filtering and/or sorting mechanism (e.g., a dropdown (for example with selectable options for System Views: Candidate/Dismissed or Public/Private saved queries)) or any currently applied manual filters.
a. AF Ablation Class I Indications subset rate and count
b. EP Specialist Evaluation rate and count by Managing Provider and Overall
c. Antiarrhythmic Prescription count by Medication including, e.g., Antiarrhythmic (AAD) for AF Rhythm Control per guidelines is limited to: Dofetilide, Dronedarone, Flecainide, Propafenone, Amiodarone, Sotalol.

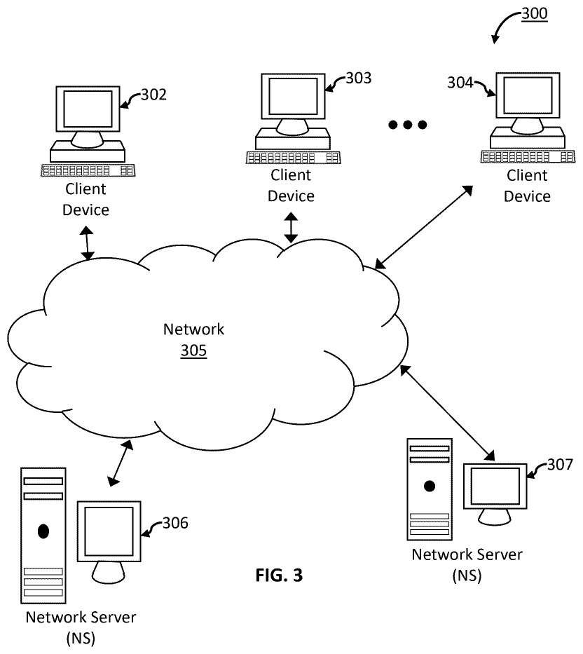

FIG. 3 depicts a block diagram of an exemplary computer-based system and platform 300 in accordance with one or more embodiments of the present disclosure. However, not all of these components may be required to practice one or more embodiments, and variations in the arrangement and type of the components may be made without departing from the spirit or scope of various embodiments of the present disclosure. According to aspects of one or more embodiments, the illustrative computing devices and the illustrative computing components of the exemplary computer-based system and platform 300 may be configured to manage a large number of members and concurrent transactions, as detailed herein. According to aspects of one or more embodiments, the exemplary computer-based system and platform 300 may be based on a scalable computer and network architecture that incorporates varies strategies for assessing the data, caching, searching, and/or database connection pooling. An example of the scalable architecture is an architecture that is capable of operating multiple servers.

According to aspects of one or more embodiments, referring to FIG. 3, client device 302, client device 303 through client device 304 (e.g., clients) of the exemplary computer-based system and platform 300 may include virtually any computing device capable of receiving and sending a message over a network (e.g., cloud network), such as network 305, to and from another computing device, such as servers 306 and 307, each other, and the like. According to aspects of one or more embodiments, the client devices 302 through 304 may be personal computers, multiprocessor systems, microprocessor-based or programmable consumer electronics, network PCs, and the like. According to aspects of one or more embodiments, one or more client devices within client devices 302 through 304 may include computing devices that typically connect using a wireless communications medium such as cell phones, smart phones, radio frequency (RF) devices, infrared (IR) devices, integrated devices combining one or more of the preceding devices, or virtually any mobile computing device, and the like. According to aspects of one or more embodiments, one or more client devices within client devices 302 through 304 may be devices that are capable of connecting using a wired or wireless communication medium such as a PDA, POCKET PC, wearable computer, a laptop, tablet, desktop computer, a netbook, a video game device, a pager, a smart phone, an ultra-mobile personal computer (UMPC), and/or any other device that is equipped to communicate over a wired and/or wireless communication medium (e.g., NFC, RFID, NBIOT, 3G, 4G, 5G, GSM, GPRS, WiFi, WiMax, CDMA, OFDM, OFDMA, LTE, satellite, ZigBee, etc.). According to aspects of one or more embodiments, one or more client devices within client devices 302 through 304 may include may run one or more applications, such as Internet browsers, mobile applications, voice calls, video games, videoconferencing, and email, among others. According to aspects of one or more embodiments, one or more client devices within client devices 302 through 304 may be configured to receive and to send web pages, and the like. According to aspects of one or more embodiments, an exemplary specifically programmed browser application of the present disclosure may be configured to receive and display graphics, text, multimedia, and the like, employing virtually any web based language, including, but not limited to Standard Generalized Markup Language (SMGL), such as HyperText Markup Language (HTML), a wireless application protocol (WAP), a Handheld Device Markup Language (HDML), such as Wireless Markup Language (WML), WMLScript, XML, JavaScript, and the like. According to aspects of one or more embodiments, a client device within client devices 302 through 304 may be specifically programmed by either Java, .Net, QT, C, C++, Python, PHP and/or other suitable programming language. In some embodiment of the device software, device control may be distributed between multiple standalone applications. According to aspects of one or more embodiments, software components/applications can be updated and redeployed remotely as individual units or as a full software suite. According to aspects of one or more embodiments, a client device may periodically report status or send alerts over text or email. According to aspects of one or more embodiments, EMR may be accessed and/or patient lists may be pushed via one or more API standards, such as, e.g., the HL7 Fast Healthcare Interoperability Resources (FHIR) standards, including the API thereof or any other suitable standards or any combination thereof. According to aspects of one or more embodiments, a client device may contain a data recorder which is remotely downloadable by the user using network protocols such as FTP, SSH, or other file transfer mechanisms. According to aspects of one or more embodiments, a client device may provide several levels of user interface, for example, advance user, standard user. According to aspects of one or more embodiments, one or more client devices within client devices 302 through 304 may be specifically programmed include or execute an application to perform a variety of possible tasks, such as, without limitation, messaging functionality, browsing, searching, playing, streaming or displaying various forms of content, including locally stored or uploaded messages, images and/or video, and/or games.

According to aspects of one or more embodiments, the exemplary network 305 may provide network access, data transport and/or other services to any computing device coupled to it. According to aspects of one or more embodiments, the exemplary network 305 may include and implement at least one specialized network architecture that may be based at least in part on one or more standards set by, for example, without limitation, Global System for Mobile communication (GSM) Association, the Internet Engineering Task Force (IETF), and the Worldwide Interoperability for Microwave Access (WiMAX) forum. According to aspects of one or more embodiments, the exemplary network 305 may implement one or more of a GSM architecture, a General Packet Radio Service (GPRS) architecture, a Universal Mobile Telecommunications System (UMTS) architecture, and an evolution of UMTS referred to as Long Term Evolution (LTE). According to aspects of one or more embodiments, the exemplary network 305 may include and implement, as an alternative or in conjunction with one or more of the above, a WiMAX architecture defined by the WiMAX forum. According to aspects of one or more embodiments and, optionally, in combination of any embodiment described above or below, the exemplary network 305 may also include, for instance, at least one of a local area network (LAN), a wide area network (WAN), the Internet, a virtual LAN (VLAN), an enterprise LAN, a layer 3 virtual private network (VPN), an enterprise IP network, or any combination thereof. According to aspects of one or more embodiments and, optionally, in combination of any embodiment described above or below, at least one computer network communication over the exemplary network 305 may be transmitted based at least in part on one of more communication modes such as but not limited to: NFC, RFID, Narrow Band Internet of Things (NBIOT), ZigBee, 3G, 4G, 5G, GSM, GPRS, WiFi, WiMax, CDMA, OFDM, OFDMA, LTE, satellite and any combination thereof. According to aspects of one or more embodiments, the exemplary network 305 may also include mass storage, such as network attached storage (NAS), a storage area network (SAN), a content delivery network (CDN) or other forms of computer or machine-readable media.

According to aspects of one or more embodiments, the exemplary server 306 or the exemplary server 307 may be a web server (or a series of servers) running a network operating system, examples of which may include but are not limited to Apache on Linux or Microsoft IIS (Internet Information Services). According to aspects of one or more embodiments, the exemplary server 306 or the exemplary server 307 may be used for and/or provide cloud and/or network computing. Although not shown in FIG. 3, According to aspects of one or more embodiments, the exemplary server 306 or the exemplary server 307 may have connections to external systems like email, SMS messaging, text messaging, ad content providers, etc. Any of the features of the exemplary server 306 may be also implemented in the exemplary server 307 and vice versa.

According to aspects of one or more embodiments, one or more of the exemplary servers 306 and 307 may be specifically programmed to perform, in non-limiting example, as authentication servers, search servers, email servers, social networking services servers, Short Message Service (SMS) servers, Instant Messaging (IM) servers, Multimedia Messaging Service (MMS) servers, exchange servers, photo-sharing services servers, advertisement providing servers, financial/banking-related services servers, travel services servers, or any similarly suitable service-base servers for users of the client devices 301 through 304.

According to aspects of one or more embodiments and, optionally, in combination of any embodiment described above or below, for example, one or more exemplary computing client devices 302 through 304, the exemplary server 306, and/or the exemplary server 307 may include a specifically programmed software module that may be configured to send, process, and receive information using a scripting language, a remote procedure call, an email, a tweet, Short Message Service (SMS), Multimedia Message Service (MMS), instant messaging (IM), an application programming interface, Simple Object Access Protocol (SOAP) methods, Common Object Request Broker Architecture (CORBA), HTTP (Hypertext Transfer Protocol), REST (Representational State Transfer), SOAP (Simple Object Transfer Protocol), MLLP (Minimum Lower Layer Protocol), or any combination thereof.

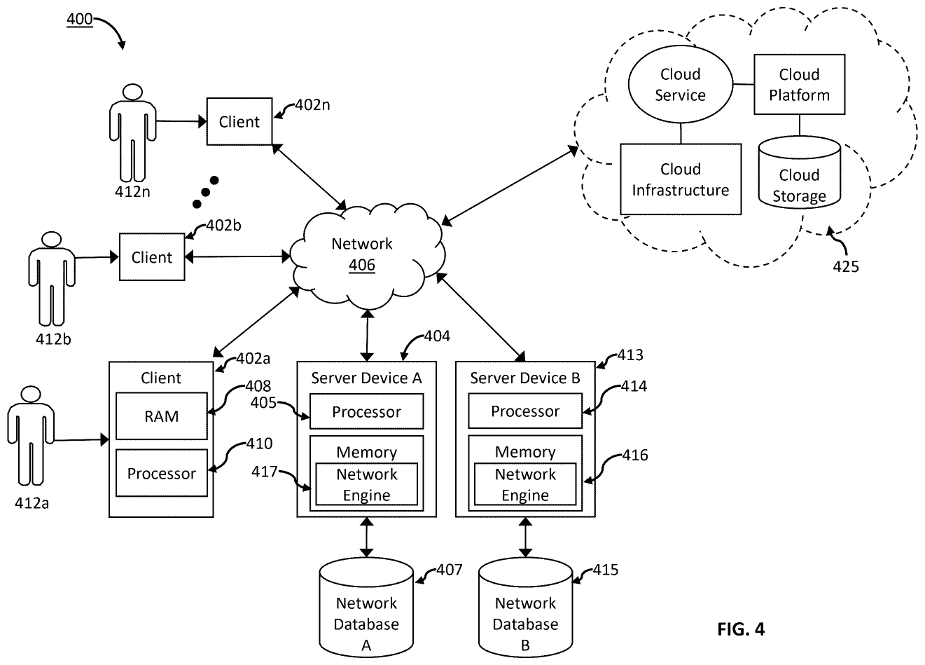

FIG. 4 depicts a block diagram of another exemplary computer-based system and platform 400 in accordance with one or more embodiments of the present disclosure. However, not all of these components may be required to practice one or more embodiments, and variations in the arrangement and type of the components may be made without departing from the spirit or scope of various embodiments of the present disclosure. According to aspects of one or more embodiments, the client device 402a, client device 402b through client device 402n shown each at least includes a computer-readable medium, such as a random-access memory (RAM) 408 coupled to a processor 410 or FLASH memory. According to aspects of one or more embodiments, the processor 410 may execute computer-executable program instructions stored in memory 408. According to aspects of one or more embodiments, the processor 410 may include a microprocessor, an ASIC, and/or a state machine. According to aspects of one or more embodiments, the processor 410 may include, or may be in communication with, media, for example computer-readable media, which stores instructions that, when executed by the processor 410, may cause the processor 410 to perform one or more steps described herein. According to aspects of one or more embodiments, examples of computer-readable media may include, but are not limited to, an electronic, optical, magnetic, or other storage or transmission device capable of providing a processor, such as the processor 410 of client device 402a, with computer-readable instructions. According to aspects of one or more embodiments, other examples of suitable media may include, but are not limited to, a floppy disk, CD-ROM, DVD, magnetic disk, memory chip, ROM, RAM, an ASIC, a configured processor, all optical media, all magnetic tape or other magnetic media, or any other medium from which a computer processor can read instructions. Also, various other forms of computer-readable media may transmit or carry instructions to a computer, including a router, private or public network, or other transmission device or channel, both wired and wireless. According to aspects of one or more embodiments, the instructions may include code from any computer-programming language, including, for example, C, C++, Visual Basic, Java, Python, Perl, JavaScript, and etc.

According to aspects of one or more embodiments, client devices 402a through 402n may also include a number of external or internal devices such as a mouse, a CD-ROM, DVD, a physical or virtual keyboard, a display, or other input or output devices. According to aspects of one or more embodiments, examples of client devices 402a through 402n (e.g., clients) may be any type of processor-based platforms that are connected to a network 406 such as, without limitation, personal computers, digital assistants, personal digital assistants, smart phones, pagers, digital tablets, laptop computers, Internet appliances, and other processor-based devices. According to aspects of one or more embodiments, client devices 402a through 402n may be specifically programmed with one or more application programs in accordance with one or more principles/methodologies detailed herein. According to aspects of one or more embodiments, client devices 402a through 402n may operate on any operating system capable of supporting a browser or browser-enabled application, such as Microsoft™, Windows™, and/or Linux. According to aspects of one or more embodiments, client devices 402a through 402n shown may include, for example, personal computers executing a browser application program such as Microsoft Corporation's Internet Explorer™, Apple Computer, Inc.'s Safari™, Mozilla Firefox, and/or Opera. According to aspects of one or more embodiments, through the member computing client devices 402a through 402n, user 412a, user 412b through user 412n, may communicate over the exemplary network 406 with each other and/or with other systems and/or devices coupled to the network 406. As shown in FIG. 4, exemplary server devices 404 and 413 may include processor 405 and processor 414, respectively, as well as memory 417 and memory 416, respectively. According to aspects of one or more embodiments, the server devices 404 and 413 may be also coupled to the network 406. According to aspects of one or more embodiments, one or more client devices 402a through 402n may be mobile clients.

According to aspects of one or more embodiments, at least one database of exemplary databases 407 and 415 may be any type of database, including a database managed by a database management system (DBMS). According to aspects of one or more embodiments, an exemplary DBMS-managed database may be specifically programmed as an engine that controls organization, storage, management, and/or retrieval of data in the respective database. According to aspects of one or more embodiments, the exemplary DBMS-managed database may be specifically programmed to provide the ability to query, backup and replicate, enforce rules, provide security, compute, perform change and access logging, and/or automate optimization. According to aspects of one or more embodiments, the exemplary DBMS-managed database may be chosen from Oracle database, IBM DB2, Adaptive Server Enterprise, FileMaker, Microsoft Access, Microsoft SQL Server, MySQL, PostgreSQL, and a NoSQL implementation. According to aspects of one or more embodiments, the exemplary DBMS-managed database may be specifically programmed to define each respective schema of each database in the exemplary DBMS, according to a particular database model of the present disclosure which may include a hierarchical model, network model, relational model, object model, or some other suitable organization that may result in one or more applicable data structures that may include fields, records, files, and/or objects. According to aspects of one or more embodiments, the exemplary DBMS-managed database may be specifically programmed to include metadata about the data that is stored.

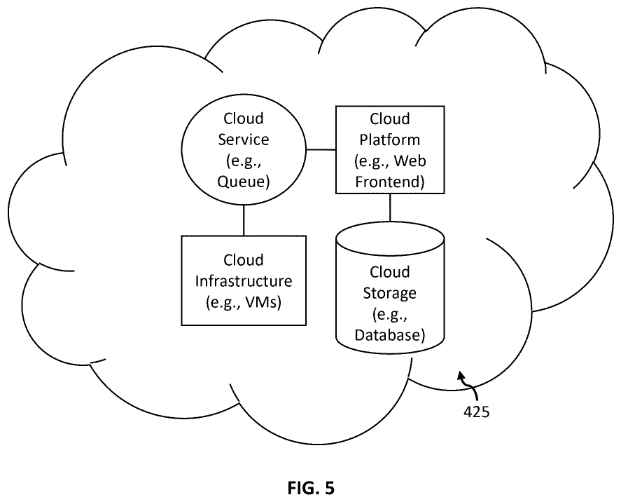

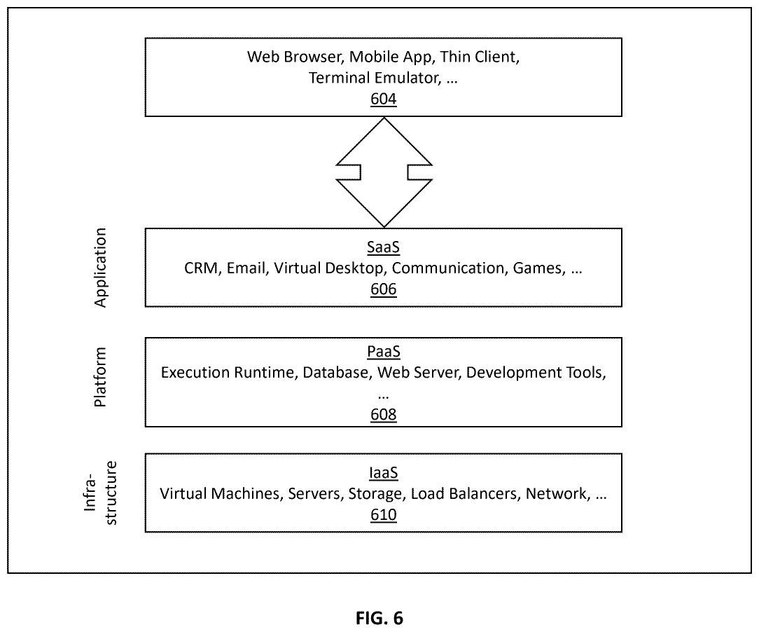

According to aspects of one or more embodiments, the exemplary inventive computer-based systems/platforms, the exemplary inventive computer-based devices, and/or the exemplary inventive computer-based components of the present disclosure may be specifically configured to operate in a cloud computing/architecture 425 such as, but not limiting to: infrastructure a service (IaaS) 610, platform as a service (PaaS) 608, and/or software as a service (SaaS) 606 using a web browser, mobile app, thin client, terminal emulator or other endpoint 604. FIGS. 5 and 6 illustrate schematics of exemplary implementations of the cloud computing/architecture(s) in which the exemplary inventive computer-based systems/platforms, the exemplary inventive computer-based devices, and/or the exemplary inventive computer-based components of the present disclosure may be specifically configured to operate.

### Examples of User Interfaces and Functions Thereof for Illustrative Cardiac Treatments

FIG. 7 depicts an exemplary Patient List Dashboard having links to patient lists across five valvular pathologies (AS, AR, MS, MR, TR) as well as heart failure and atrial fibrillation. Included with the link for each patient list may be the total number of active patients currently on the list. Dynamic patient relevance platform 100 defines a patient as ‘active’ if that patient may be not expired, not palliative care (defined as 2 or more palliative care encounters/orders or Diagnosis Code Z51.5) and not lost to follow up (at least one encounter, procedure, echo, manual entry, admission or CV diagnosis within the last 300 days).

Though these lists may be comprehensive, all available data points for Dynamic patient relevance platform 100 patients may be found within the data source(s) 102 of the dynamic patient relevance platform 100.

In the top right corner of the patient list dashboard, there may be a filter to toggle between All and Unreviewed (All patients on the lists or only the patients with new, unreviewed list information.

This dashboard having 3 major sections, described below. A detailed description of List criteria follows. When considering patients for inclusion within the VHD related Patient Lists, the last known or clearly stated valve severity as extracted by the dynamic patient relevance platform 100 NLP from within the echocardiogram may be utilized if the most recent echocardiogram does not include a clearly stated severity.
a. High Priority Patients: provides a list of patients diagnosed with significant disease that have not yet been evaluated by a specialist. Additional criteria used by the software to determine inclusion on the list for each disease state may be displayed prominently below each disease state icon. High priority lists may be available for five valve pathologies (AS, AR, MS, MR, TR) in addition to HFrEF and AF GDMT Eligible.
b. Candidates for Intervention: lists of patients who meet guideline(s) for a given treatment/therapy and/or inclusion/exclusion criteria for a given clinical trial. For each candidate patient list, the guideline(s) utilized to placed patients on the list and the published guidelines and/or literature used to inform the guideline(s) may be visibly displayed within the ‘Screening Reference’ tab. Candidate patient lists may be available for AS AVR, AR AVR, MS MVR, MR MVR, TEER, PA Sensor, ICD, CRTD, LAAC and AF Ablation.
c. Disease Management: lists of patients with stated disease. These lists include severe and moderate-to-severe diagnoses for the five valve pathologies, HFrEF and/or HFpEF patients, AF GDMT Eligible patients, Diagnostic Precision (severe AS hemodynamic value without a severe AS diagnosis) and Disease Progression (Moderate AS) lists. A brief description of the predictive algorithm lists may be found below, and a detailed description may be found within a link in the UI.
   i. Diagnostic Precision module retrospectively identifies potential discrepancies between the physician diagnosis and guideline-indicated measurements to assess aortic stenosis. Diagnostic Precision function may be not intended to be used on a stand-alone basis for diagnostic purposes.
   ii. Disease Progression module assists physicians in prioritizing moderate aortic stenosis patients for follow-up; the software assigns a score to each patient with higher scores reflecting a higher likelihood that the aortic stenosis has progressed to severe.

When a List may be clicked from the Patient List Dashboard, the user may be redirected to that Patient List. This list may include filters innate to the name of the report, i.e., Severe AS may have ‘Valve Pathology’ of ‘AS’ and ‘Severity’ of ‘Severe’ automatically selected.

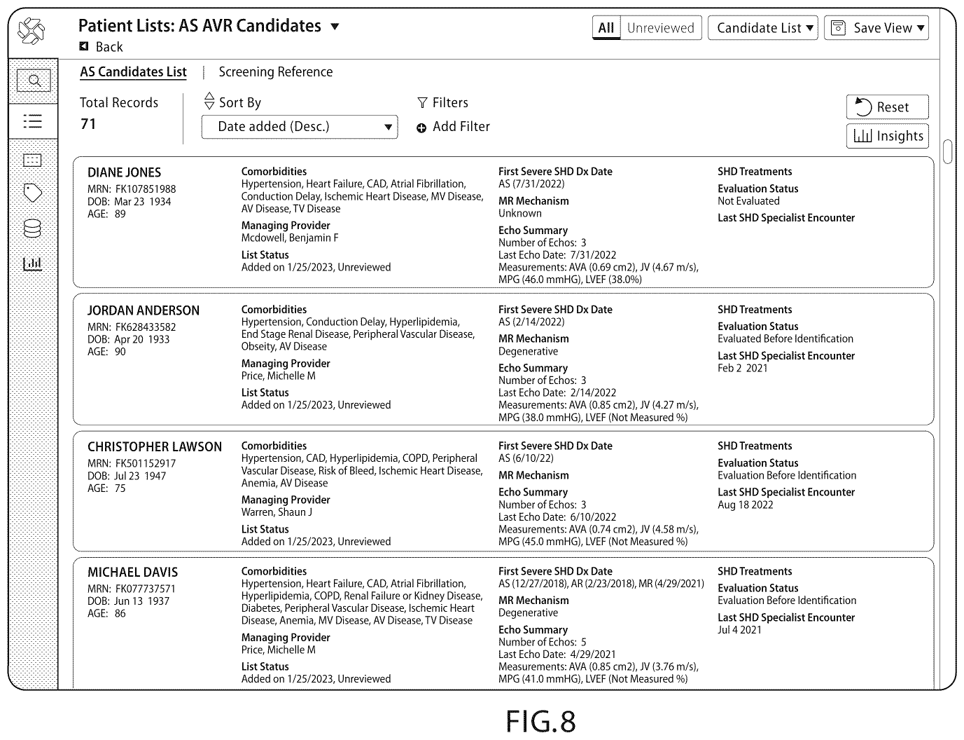

FIG. 8—Patient Lists display the number of patients meeting clinical practice criteria consistent with significant disease. Within a patient list, a user may add filters, sort by ascending/descending order, save custom list queries and create public and private list queries.

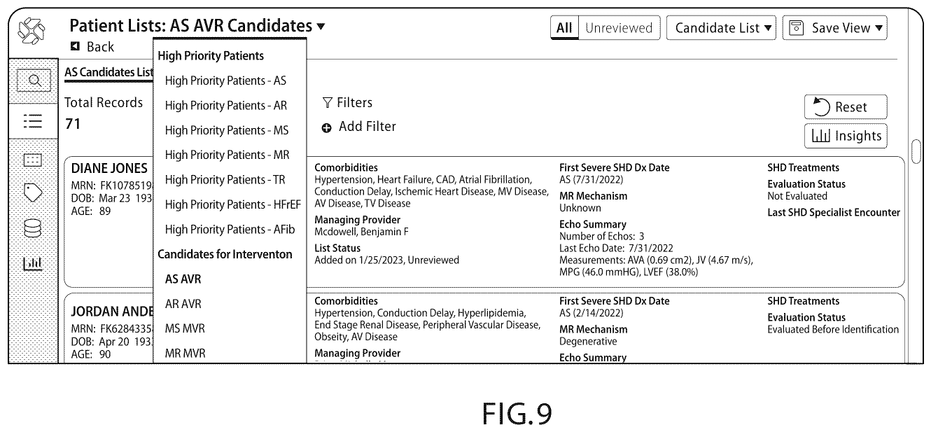

At the top of any given Patient List may be a dropdown (FIG. 9) may include lists visible within the Patient List Dashboard.

This dropdown (top left of image above) may show all available Patient Lists and allows the user to navigate to any Patient List without returning to the dashboard. Within a given list, the blue Back Arrow in the top left corner allows the user to return to the previous page.

Moving from left to right across the top of a list, the ‘Sort By’ allows the user to sort in ascending and descending order of a variety of datapoints.

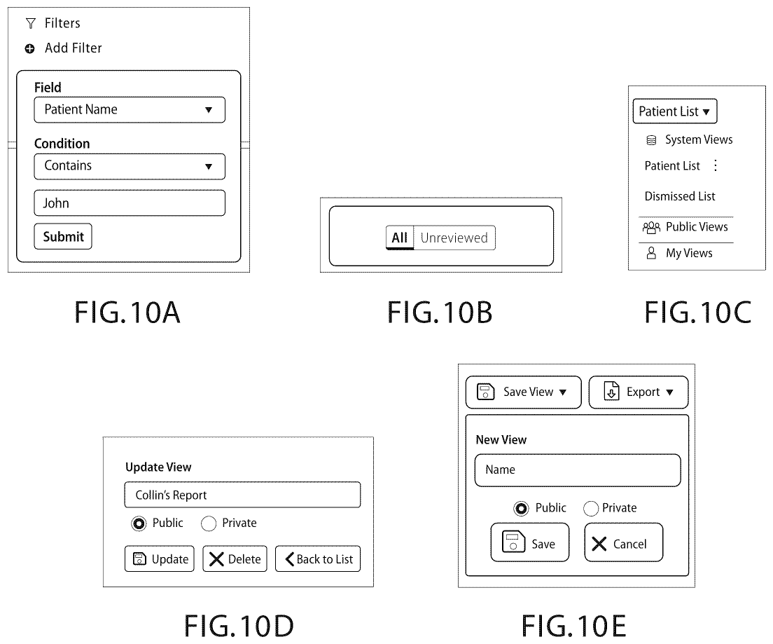

The Filters/Add Filter button (FIG. 10A) allows a user to add filters to the list of patients. The options available in the Field dropdown may be unique to each list.

Like the Patient List Dashboard, the Patient Lists may include a filter (FIG. 10B) to view All patients or only patients who may be Unreviewed as indicated by the end user. When viewing a select Patient List, “Unreviewed” may display patients who may be Unreviewed for the specific Patient List.

The List Query dropdown (FIG. 10C) having System Views created by dynamic patient relevance platform 100, Public Views and Private Views. The user may be able to see their own private queries (My Views), as well as public queries created by other users

Clicking on the three ellipses next to a List query name opens a dialog (FIG. 10D) that allows a user to update the name of a list query without changing any characteristics of it and allows for deletion of a query created by the specific end user.

The Save View button (FIG. 10E) may allow a user to save updates they made to the list, the filters or the general sorting. If none of those actions were completed, this button may be disabled.

List queries may be saved as Public or Private. Public queries may be visible to all other users, Private queries may be visible only to the user who created and saved it. Additionally, Public queries created by other users may be edited and saved as other queries if the visible columns or name of the query may be changed and saved.

The Reset button may remove any filters applied that have not been saved to the current view or a new view. It may not remove all filters, only those not saved to the query.

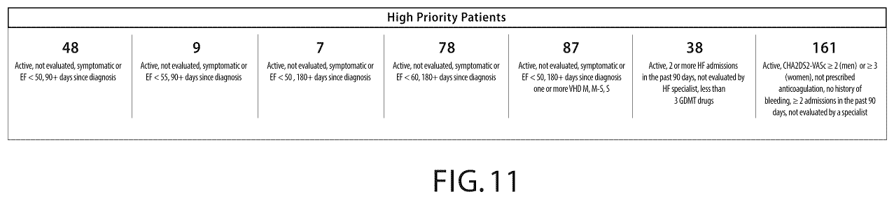

FIG. 11 illustrates exemplary High Priority Patient Lists may be designed to draw attention to patients for secondary review and includes patients who may be active within a system (not expired, not lost to follow up, not palliative care), and meet select criteria which may be defined below. Patients may be excluded from the AS, AR, MS, MR and TR lists if treated via procedure for the disease specified, or from the AF list if treated via left atrial appendage closure (LAAC). Where L VEF may be inclusion criteria this may be based upon the most recent LVEF documented within Dynamic patient relevance platform 100.

High Priority Patient List criteria may be defined as the following:
a. High Priority AS: Active, not evaluated by an SH specialist, symptomatic or EF<50, 90+ days since first Severe diagnosis.
b. High Priority AR: Active, not evaluated by an SH specialist, symptomatic or EF<55, 90+ days since first Severe diagnosis.
c. High Priority MS: Active, not evaluated by an SH specialist, symptomatic or EF<50, 180+ days since first Severe diagnosis.
d. High Priority MR: Active, not evaluated by an SH specialist, symptomatic or EF<60, 180+ days since first Severe diagnosis.
e. High Priority TR: Active, not evaluated by an SH specialist, symptomatic or EF<50, 180+ days since first Severe diagnosis, one or more additional valves diagnosed Moderate, Moderate-to-Severe or Severe
f. High Priority HFrEF: Active, 2 or more HF admissions in the past 90 days, not evaluated by HF specialist, less than 3 GDMT drugs prescribed (see HFrEF List for further detail)
g. High Priority AF: Active, CHA₂DS₂-VASc≥2 (men) or ≥3 (women), not prescribed anticoagulation, no history of bleeding, ≥2 admissions in the past 90 days, not evaluated by an EP specialist.

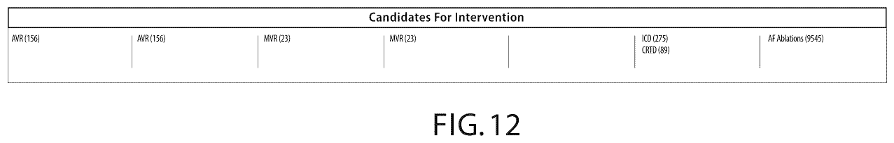

FIG. 12 illustrates exemplary candidate patient lists and related insights may include potential patients that might be prioritized for secondary review and/or intervention based on published guidelines. Each Patient list includes a Candidate patient list and Dismissed list, including a Screening Reference which contains details on guidelines and indications used to identify potential patients for that list. The insights may include additional detail in the form of charts and graphs with further analysis. ‘Inactive’ patients or patients meeting Dynamic patient relevance platform 100 palliative/hospice or ‘Lost to Follow Up’ criteria and expired patients may be not included within Candidate Patient Lists.

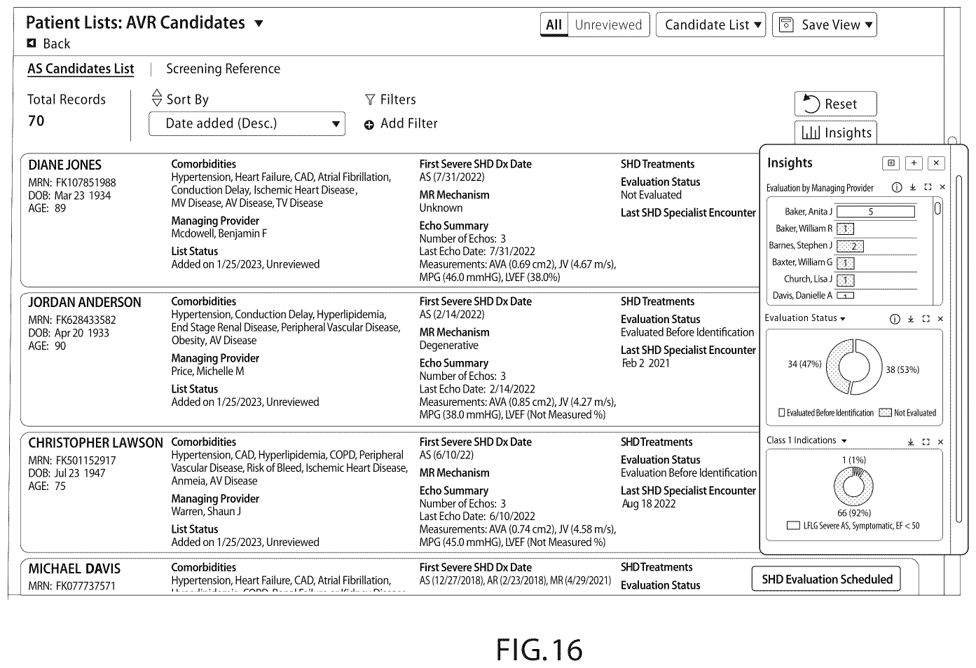

Visible within select Candidate and Patient Lists may be Insights as illustrated, for example in FIG. 16, or analytic graphics which provide a visual representation of the selected or filtered patient subset. These Insights may be viewed by clicking on the “Insights” button in the upper right of the selected Patient List.

A variety of functions may be available within the Insights by selecting the respective buttons below. The + button allows the user to Expand the Insights section for viewing in a larger format. Insights may be Positioned at the top, bottom, left or right of the page by clicking the button with bisecting horizontal and vertical arrows.

Additionally, individual reports may be available for Download (icon of a down pointing arrow), expanded by Focus mode (square icon), or removed from view by clicking the Remove (X icon) button. Select Insights also may include an Information icon (i), which when hovered may provide additional detail or definition related to the subset may be included within the graphic.

Any Insights graphs which have been previously Removed from a select Patient List may be added at a future time by clicking the + Add Insights button at the bottom of the Insights panel.

The ‘Add/Dismiss’ Lists tab of the Action Bar displays the status of all Lists for a patient and allows the user to add and dismiss patients from those lists. The lists may be organized into the same categories visible on the Patient List Dashboard (High Priority Patients, Candidates for Intervention, Disease Management).

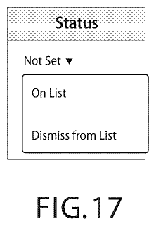

The state of each list may be displayed through a drop-down (FIG. 17) may including three options: Not Set, On List and Dismiss from List.

Not Set means that the patient may be not on that list. The patient has not met Dynamic patient relevance platform 100 defined criteria for inclusion on stated Patient list.

On List indicates that the patient may be currently on the stated List. A user may also add a patient to any list by selecting this option from the dropdown list manually.

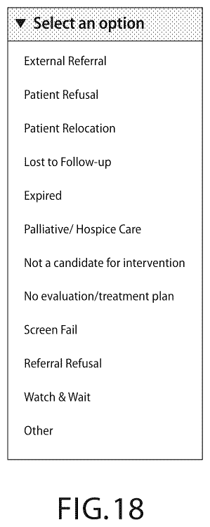

Dismiss from List (FIG. 18) indicates that the patient has been dismissed from the List by a user.

If new data that may be entered by the navigator or EMR data extracted from the nightly refresh renders a patient ineligible for a list because they no longer fit the criteria, they may be removed from the list, not dismissed. Therefore, the List Status may revert to Not Set.

When dismissing a patient from the List, a user must enter a Dismiss Reason and Reassess Condition.

The available Dismissal Reasons may be as follows:
a. External Referral
b. Patient Refusal
c. Patient Relocation
d. Lost to Follow-up
e. Expired
f. Palliative/Hospice Care
g. Not a candidate for intervention
h. No evaluation/treatment plan
i. Screen Fail
j. Referral Refusal
k. Watch & Wait
l. Other

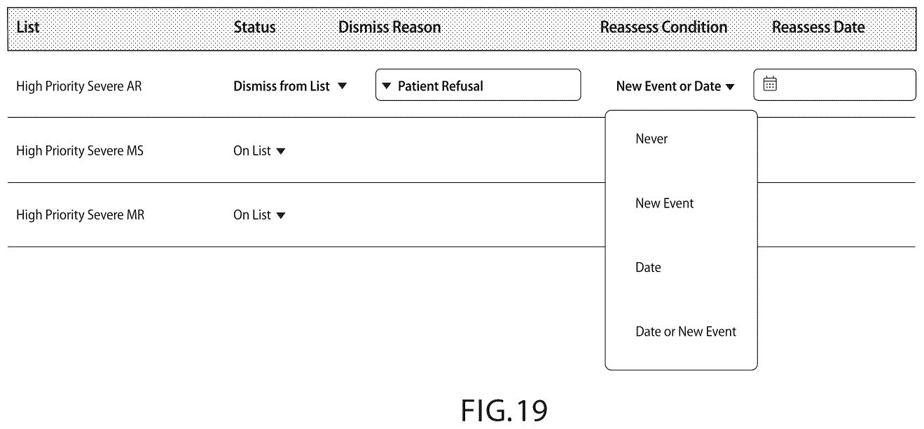

Reassess Condition (FIG. 19) allows users to choose the actions which may revert a patient back to “On List” or choose the Never option if they wish to permanently dismiss the patient. Only future dated “Reassess Dates” may be visible within the Scheduling Manager.

The available Reassess Condition options may be as follows:
a. Never
b. New Event
c. Date
d. Date or New Event

Patients dismissed with a Reassess Condition of “New Event” may revert to “On List” as Unreviewed when below criteria may be met, assuming the patient still meets Patient/Candidate patient list specific criteria and may no longer be categorized as dismissed within the Add/Dismiss section of the Patient Review page. If a patient no longer meets patient list criteria and the dismissal type was date based, the patient may be visible within the Schedule Manager until the Reassess Date has been reached.

TABLE 9
Exemplary events that trigger a patient to
return to Patient List as Unreviewed

| | Events that Trigger Dismissed > |
|---|---|
| Lists | On Patient List |
| Structural Heart Disease Lists | New echo with severe diagnosis for any |
| AS, AR, MS, MR, TR High | valve pathology (not only the valve |
| Priority, Severe or Mod-Severe | related to the specific list). ONLY |
| Disease Management, AVR, | echo diagnosis, not CDW or problem |
| TEER | list diagnosis. New severe diagnosis or |
| | subsequent severe diagnosis after |
| | initial placement on list. |
| Heart Failure Lists | New echo with an EF ≤40% |
| HFrEF High Priority, HFrEF, | |
| HFpEF, PA Sensor, ICD, CRTD | |
| Atrial Fibrillation Lists | A different AF diagnosis |
| AF High Priority, AF GDMT, | (I.e., Previous diagnosis before |
| LAAC, AF Ablation | marked as reviewed was Unspecified. |
| | New diagnosis may be Persistent; Or |
| | was Paroxysmal now Permanent) |
| | AND a documented EKG. |

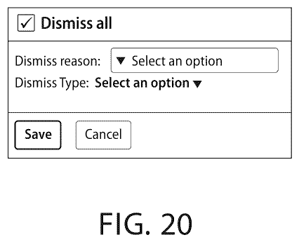

The user may also dismiss all Lists for patient, for example, if the patient expired, and their expiration may be not recorded in the EMR. To dismiss all Lists, scroll to the bottom of the Lists page (FIG. 20).

To complete the transaction, check the ‘Dismiss All’ checkbox, select a Dismiss Reason from the dropdown, select a Reassess Date (if desired) and click Save.

When Reassess Date events may be clicked on in the Scheduling Manager, the user may be redirected to the “Add/Dismiss Lists” section of the Patient Review page. From there the user has three options.
a. Update the “Reassess Date” to a future date if it may be desired for the patient to remain Dismissed from said list but remain following. Click save at the top of the list after editing.
b. Update the “Reassess Condition” to “Event” if it may be desired for the patient to remain Dismissed from said list but continue following without a predetermined follow-up date. Click save at the top of the list after editing.
c. Update the “Reassess Condition” to “Never.” Click save at the top of the list after editing.

It is understood that at least one aspect/functionality of various embodiments described herein can be performed in real-time and/or dynamically. As used herein, the term “real-time” is directed to an event/action that can occur instantaneously or almost instantaneously in time when another event/action has occurred. For example, the “real-time processing,” “real-time computation,” and “real-time execution” all pertain to the performance of a computation during the actual time that the related physical process (e.g., a user interacting with an application on a mobile device) occurs, in order that results of the computation can be used in guiding the physical process.

As used herein, the term “dynamically” and term “automatically,” and their logical and/or linguistic relatives and/or derivatives, mean that certain events and/or actions can be triggered and/or occur without any human intervention. According to aspects of one or more embodiments, events and/or actions in accordance with the present disclosure can be in real-time and/or based on a predetermined periodicity of at least one of: nanosecond, several nanoseconds, millisecond, several milliseconds, second, several seconds, minute, several minutes, hourly, several hours, daily, several days, weekly, monthly, etc.

As used herein, the term “runtime” corresponds to any behavior that is dynamically determined during an execution of a software application or at least a portion of software application.

According to aspects of one or more embodiments, exemplary inventive, specially programmed computing systems and platforms with associated devices are configured to operate in the distributed network environment, communicating with one another over one or more suitable data communication networks (e.g., the Internet, satellite, etc.) and utilizing one or more suitable data communication protocols/modes such as, without limitation, IPX/SPX, X.25, AX.25, AppleTalk™, TCP/IP (e.g., HTTP), near-field wireless communication (NFC), RFID, Narrow Band Internet of Things (NBIOT), 3G, 4G, 5G, GSM, GPRS, WiFi, WiMax, CDMA, satellite, ZigBee, and other suitable communication modes.

According to aspects of one or more embodiments, the NFC can represent a short-range wireless communications technology in which NFC-enabled devices are “swiped,” “bumped,” “tap” or otherwise moved in close proximity to communicate. According to aspects of one or more embodiments, the NFC could include a set of short-range wireless technologies, typically requiring a distance of 10 cm or less. According to aspects of one or more embodiments, the NFC may operate at 13.56 MHz on ISO/IEC 18000-3 air interface and at rates ranging from 106 kbit/s to 424 kbit/s. According to aspects of one or more embodiments, the NFC can involve an initiator and a target; the initiator actively generates an RF field that can power a passive target. In some embodiment, this can enable NFC targets to take very simple form factors such as tags, stickers, key fobs, or cards that do not require batteries. According to aspects of one or more embodiments, the NFC's peer-to-peer communication can be conducted when a plurality of NFC-enable devices (e.g., smartphones) within close proximity of each other.

The material disclosed herein may be implemented in software or firmware or a combination of them or as instructions stored on a machine-readable medium, which may be read and executed by one or more processors. A machine-readable medium may include any medium and/or mechanism for storing or transmitting information in a form readable by a machine (e.g., a computing device). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others.

As used herein, the terms “computer engine” and “engine” identify at least one software component and/or a combination of at least one software component and at least one hardware component which are designed/programmed/configured to manage/control other software and/or hardware components (such as the libraries, software development kits (SDKs), objects, etc.).

Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. According to aspects of one or more embodiments, the one or more processors may be implemented as a Complex Instruction Set Computer (CISC) or Reduced Instruction Set Computer (RISC) processors; x86 instruction set compatible processors, multi-core, or any other microprocessor or central processing unit (CPU). In various implementations, the one or more processors may be dual-core processor(s), dual-core mobile processor(s), and so forth.

Computer-related systems, computer systems, and systems, as used herein, include any combination of hardware and software. Examples of software may include software components, programs, applications, operating system software, middleware, firmware, software modules, routines, subroutines, functions, methods, procedures, software interfaces, application program interfaces (API), instruction sets, computer code, computer code segments, words, values, symbols, or any combination thereof. Determining whether an embodiment is implemented using hardware elements and/or software elements may vary in accordance with any number of factors, such as desired computational rate, power levels, heat tolerances, processing cycle budget, input data rates, output data rates, memory resources, data bus speeds and other design or performance constraints.

One or more aspects of at least one embodiment may be implemented by representative instructions stored on a machine-readable medium which represents various logic within the processor, which when read by a machine causes the machine to fabricate logic to perform the techniques described herein. Such representations, known as “IP cores,” may be stored on a tangible, machine readable medium and supplied to various customers or manufacturing facilities to load into the fabrication machines that make the logic or processor. Of note, various embodiments described herein may, of course, be implemented using any appropriate hardware and/or computing software languages (e.g., C++, Objective-C, Swift, Java, JavaScript, Python, Perl, QT, etc.).

According to aspects of one or more embodiments, one or more of illustrative computer-based systems or platforms of the present disclosure may include or be incorporated, partially or entirely into at least one personal computer (PC), laptop computer, ultra-laptop computer, tablet, touch pad, portable computer, handheld computer, palmtop computer, personal digital assistant (PDA), cellular telephone, combination cellular telephone/PDA, television, smart device (e.g., smart phone, smart tablet or smart television), mobile internet device (MID), messaging device, data communication device, and so forth.

As used herein, term “server” should be understood to refer to a service point which provides processing, database, and communication facilities. By way of example, and not limitation, the term “server” can refer to a single, physical processor with associated communications and data storage and database facilities, or it can refer to a networked or clustered complex of processors and associated network and storage devices, as well as operating software and one or more database systems and application software that support the services provided by the server. Cloud servers are examples.

According to aspects of one or more embodiments, as detailed herein, one or more of the computer-based systems of the present disclosure may obtain, manipulate, transfer, store, transform, generate, and/or output any digital object and/or data unit (e.g., from inside and/or outside of a particular application) that can be in any suitable form such as, without limitation, a file, a contact, a task, an email, a message, a map, an entire application (e.g., a calculator), data points, and other suitable data. According to aspects of one or more embodiments, as detailed herein, one or more of the computer-based systems of the present disclosure may be implemented across one or more of various computer platforms such as, but not limited to: (1) FreeBSD, NetBSD, OpenBSD; (2) Linux; (3) Microsoft Windows™; (4) OpenVMS™; (5) OS X (MacOS™); (6) UNIX™; (7) Android; (8) iOS™; (9) Embedded Linux; (10) Tizen™; (11) WebOS™; (12) Adobe AIR™; (13) Binary Runtime Environment for Wireless (BREW™); (14) Cocoa™ (API); (15) Cocoa™ Touch; (16) Java™ Platforms; (17) JavaFX™; (18) QNX™; (19) Mono; (20) Google Blink; (21) Apple WebKit; (22) Mozilla Gecko™; (23) Mozilla XUL; (24) .NET Framework; (25) Silverlight™; (26) Open Web Platform; (27) Oracle Database; (28) Qt™; (29) SAP NetWeaver™; (30) Smartface™; (31) Vexi™; (32) Kubernetes™ and (33) Windows Runtime (WinRT™) or other suitable computer platforms or any combination thereof. According to aspects of one or more embodiments, illustrative computer-based systems or platforms of the present disclosure may be configured to utilize hardwired circuitry that may be used in place of or in combination with software instructions to implement features consistent with principles of the disclosure. Thus, implementations consistent with principles of the disclosure are not limited to any specific combination of hardware circuitry and software. For example, various embodiments may be embodied in many different ways as a software component such as, without limitation, a stand-alone software package, a combination of software packages, or it may be a software package incorporated as a “tool” in a larger software product.

For example, exemplary software specifically programmed in accordance with one or more principles of the present disclosure may be downloadable from a network, for example, a website, as a stand-alone product or as an add-in package for installation in an existing software application. For example, exemplary software specifically programmed in accordance with one or more principles of the present disclosure may also be available as a client-server software application, or as a web-enabled software application. For example, exemplary software specifically programmed in accordance with one or more principles of the present disclosure may also be embodied as a software package installed on a hardware device.

According to aspects of one or more embodiments, illustrative computer-based systems or platforms of the present disclosure may be configured to handle numerous concurrent users that may be, but is not limited to, at least 100 (e.g., but not limited to, 100-999), at least 1,000 (e.g., but not limited to, 1,000-9,999), at least 10,000 (e.g., but not limited to, 10,000-99,999), at least 100,000 (e.g., but not limited to, 100,000-999,999), at least 1,000,000 (e.g., but not limited to, 1,000,000-9,999,999), at least 10,000,000 (e.g., but not limited to, 10,000,000-99,999,999), at least 100,000,000 (e.g., but not limited to, 100,000,000-999,999,999), at least 1,000,000,000 (e.g., but not limited to, 1,000,000,000-999,999,999,999), and so on.

According to aspects of one or more embodiments, illustrative computer-based systems or platforms of the present disclosure may be configured to output to distinct, specifically programmed graphical user interface implementations of the present disclosure (e.g., a desktop, a web app., etc.). In various implementations of the present disclosure, a final output may be displayed on a displaying screen which may be, without limitation, a screen of a computer, a screen of a mobile device, or the like. In various implementations, the display may be a holographic display. In various implementations, the display may be a transparent surface that may receive a visual projection. Such projections may convey various forms of information, images, or objects. For example, such projections may be a visual overlay for a mobile augmented reality (MAR) application.

According to aspects of one or more embodiments, illustrative computer-based systems or platforms of the present disclosure may be configured to be utilized in various applications which may include, but not limited to, gaming, mobile-device games, video chats, video conferences, live video streaming, video streaming and/or augmented reality applications, mobile-device messenger applications, and others similarly suitable computer-device applications.

As used herein, the term “mobile electronic device,” or the like, may refer to any portable electronic device that may or may not be enabled with location tracking functionality (e.g., MAC address, Internet Protocol (IP) address, or the like). For example, a mobile electronic device can include, but is not limited to, a mobile phone, Personal Digital Assistant (PDA), Blackberry™, Pager, Smartphone, or any other reasonable mobile electronic device.

As used herein, terms “proximity detection,” “locating,” “location data,” “location information,” and “location tracking” refer to any form of location tracking technology or locating method that can be used to provide a location of, for example, a particular computing device, system or platform of the present disclosure and any associated computing devices, based at least in part on one or more of the following techniques and devices, without limitation: accelerometer(s), gyroscope(s), Global Positioning Systems (GPS); GPS accessed using Bluetooth™; GPS accessed using any reasonable form of wireless and non-wireless communication; WiFi™ server location data; Bluetooth™ based location data; triangulation such as, but not limited to, network based triangulation, WiFi™ server information based triangulation, Bluetooth™ server information based triangulation; Cell Identification based triangulation, Enhanced Cell Identification based triangulation, Uplink-Time difference of arrival (U-TDOA) based triangulation, Time of arrival (TOA) based triangulation, Angle of arrival (AOA) based triangulation; techniques and systems using a geographic coordinate system such as, but not limited to, longitudinal and latitudinal based, geodesic height based, Cartesian coordinates based; Radio Frequency Identification such as, but not limited to, Long range RFID, Short range RFID; using any form of RFID tag such as, but not limited to active RFID tags, passive RFID tags, battery assisted passive RFID tags; or any other reasonable way to determine location. For ease, at times the above variations are not listed or are only partially listed; this is in no way meant to be a limitation.

As used herein, terms “cloud,” “Internet cloud,” “cloud computing,” “cloud architecture,” and similar terms correspond to at least one of the following: (1) a large number of computers connected through a real-time communication network (e.g., Internet); (2) providing the ability to run a program or application on many connected computers (e.g., physical machines, virtual machines (VMs)) at the same time; (3) network-based services, which appear to be provided by real server hardware, and are in fact served up by virtual hardware (e.g., virtual servers), simulated by software running on one or more real machines (e.g., allowing to be moved around and scaled up (or down) on the fly without affecting the end user).

According to aspects of one or more embodiments, the illustrative computer-based systems or platforms of the present disclosure may be configured to securely store and/or transmit data by utilizing one or more of encryption techniques (e.g., private/public key pair, Triple Data Encryption Standard (3DES), block cipher algorithms (e.g., IDEA, RC2, RC5, CAST and Skipjack), cryptographic hash algorithms (e.g., MD5, RIPEMD-160, RTRO, SHA-1, SHA-2, Tiger (TTH), WHIRLPOOL, RNGs).

As used herein, the term “user” shall have a meaning of at least one user. According to aspects of one or more embodiments, the terms “user”, “subscriber” “consumer” or “customer” should be understood to refer to a user of an application or applications as described herein and/or a consumer of data supplied by a data provider. By way of example, and not limitation, the terms “user” or “subscriber” can refer to a person who receives data provided by the data or service provider over the Internet in a browser session or can refer to an automated software application which receives the data and stores or processes the data.

The aforementioned examples are, of course, illustrative and not restrictive.

At least some aspects of the present disclosure will now be described with reference to the following numbered clauses.

Clause 1. A method including: determining, by at least one processor, a condition-related criteria associated with diagnosis or treatment or both of a condition; determining, by the at least one processor, a set of condition-related criteria logic rules derived from the condition-related criteria, where the set of condition-related criteria logic rules include logic-based rules that define clinically significant information for the condition; where the set of condition-related criteria logic rules include: at least one health data type associated with the clinically significant information for the condition, and at least one threshold for the at least one health data type; determining, by the at least one processor, at least one clinical data parser configured to parse data associated with the at least one health data type so as to extract the clinically significant information from the data; inputting, by the at least one processor, a plurality of electronic medical record associated with a plurality of patients into the at least one clinical data parser to extract the clinically significant information from each electronic medical record; generating, by the at least one processor, a plurality of normalized patient data objects for the plurality of patients by mapping the clinically significant information extracted from each electronic medical record to a normalized patient data structure; applying, by the at least one processor, the set of condition-related criteria logic rules and the at least one threshold to the plurality of normalized patient data objects to produce, for each normalized patient data object, a clinical relevance indicator indicating a degree of relevance of the clinically significant information of each patient to the condition; and generating, by the at least one processor, a dashboard user interface to display a list of the plurality of patients ordered according to relevance to the condition based at least in part on the degree of relevance of the clinically significant information of each patient to the condition; where the dashboard user interface presents each normalized patient data structure and each electronic medical record associated with each patient.

Clause 2. The method as recited in clause 1, where the condition is associated with at least one treatment including at least one of: Left Atrial Appendage Closure (LAAC), pulmonary artery (PA) sensor, mitral transcatheter edge-to-edge repair (TEER), aortic valve replacement (AVR), mitral valve replacement (MVR), implantable cardioverter-defibrillator (ICD), cardiac resynchronization therapy (CRTD), or atrial fibrillation (AF) ablation.

Clause 3. The method as recited in clause 1, where the at least one clinical data parser includes at least one severity natural language processor configured to process echocardiogram reports and extract indications of severity of a cardiac condition.

Clause 4. The method as recited in clause 1, where the at least one clinical data parser is configured to identify and extract at least one diagnostic code from the plurality of electronic medical records.

Clause 5. The method as recited in clause 1, where the at least one clinical data parser is configured to determine at least one admissions statistic associated with at least one patient of the plurality of patients based at least in part on at least one time associated with at least one entry in at least one electronic medical of the plurality of electronic medical records.

Clause 6. The method as recited in clause 1, where the at least one clinical data parser is configured to identify and extract at least one laboratory test result; and where the at least one threshold includes at least one threshold value associated with the at least one laboratory test result, the at least one threshold value defining relevance of the at least one laboratory test result to the condition.

Clause 7. The method as recited in clause 1, further including: utilizing, by the at least one processor, at least one condition-related criteria machine learning model to ingest the condition-related criteria and, based at least in part on a plurality of trained condition-related criteria machine learning model parameters, output: the at least one health data type associated with the clinically significant information for the condition, and the at least one threshold for the at least one health data type; and generating, by the at least one processor, the set of logic rules based at least in part on: the at least one health data type associated with the clinically significant information for the condition, and the at least one threshold for the at least one health data type.

Clause 8. A system including: at least one processor which, upon execution of instructions, is configured to: determine a condition-related criteria associated with diagnosis or treatment or both of a condition; determine a set of condition-related criteria logic rules derived from the condition-related criteria, where the set of condition-related criteria logic rules include logic-based rules that define clinically significant information for the condition; where the set of condition-related criteria logic rules include: at least one health data type associated with the clinically significant information for the condition, and at least one threshold for the at least one health data type; determine at least one clinical data parser configured to parse data associated with the at least one health data type so as to extract the clinically significant information from the data; input a plurality of electronic medical record associated with a plurality of patients into the at least one clinical data parser to extract the clinically significant information from each electronic medical record; generate a plurality of normalized patient data objects for the plurality of patients by mapping the clinically significant information extracted from each electronic medical record to a normalized patient data structure; apply the set of condition-related criteria logic rules and the at least one threshold to the plurality of normalized patient data objects to produce, for each normalized patient data object, a clinical relevance indicator indicated a degree of relevance of the clinically significant information of each patient to the condition; and generate a dashboard user interface to display a list of the plurality of patients ordered according to relevance to the condition based at least in part on the degree of relevance of the clinically significant information of each patient to the condition; where the dashboard user interface presents each normalized patient data structure and each electronic medical record associated with each patient.

Clause 9. The system as recited in clause 8, where the condition is associated with at least one treatment including at least one of: Left Atrial Appendage Closure (LAAC), pulmonary artery (PA) sensor, mitral transcatheter edge-to-edge repair (TEER), aortic valve replacement (AVR), mitral valve replacement (MVR), implantable cardioverter-defibrillator (ICD), cardiac resynchronization therapy (CRTD), or atrial fibrillation (AF) ablation.

Clause 10. The system as recited in clause 8, where the at least one clinical data parser includes at least one severity natural language processor configured to process echocardiogram reports and extract indications of severity of a cardiac condition.

Clause 11. The system as recited in clause 8, where the at least one clinical data parser is configured to identify and extract at least one diagnostic code from the plurality of electronic medical records.

Clause 12. The system as recited in clause 8, where the at least one clinical data parser is configured to determine at least one admissions statistic associated with at least one patient of the plurality of patients based at least in part on at least one time associated with at least one entry in at least one electronic medical of the plurality of electronic medical records.

Clause 13. The system as recited in clause 8, where the at least one clinical data parser is configured to identify and extract at least one laboratory test result; and where the at least one threshold includes at least one threshold value associated with the at least one laboratory test result, the at least one threshold value defining relevance of the at least one laboratory test result to the condition.

Clause 14. The system as recited in clause 8, where the at least one processor is further configured to: utilize at least one condition-related criteria machine learning model to ingest the condition-related criteria and, based at least in part on a plurality of trained condition-related criteria machine learning model parameters, output: the at least one health data type associated with the clinically significant information for the condition, and the at least one threshold for the at least one health data type; and generate the set of logic rules based at least in part on: the at least one health data type associated with the clinically significant information for the condition, and the at least one threshold for the at least one health data type.

Clause 15. A non-transitory computer-readable medium having instructions stored thereon, the instructions being configured to, upon execution, cause at least one processor to perform steps including: obtaining a condition-related criteria associated with diagnosis or treatment or both of a condition; determining a set of condition-related criteria logic rules derived from the condition-related criteria, where the set of condition-related criteria logic rules include logic-based rules that define clinically significant information for the condition; where the set of condition-related criteria logic rules include: at least one health data type associated with the clinically significant information for the condition, and at least one threshold for the at least one health data type; determining at least one clinical data parser configured to parse data associated with the at least one health data type so as to extract the clinically significant information from the data; inputting a plurality of electronic medical record associated with a plurality of patients into the at least one clinical data parser to extract the clinically significant information from each electronic medical record; generating a plurality of normalized patient data objects for the plurality of patients by mapping the clinically significant information extracted from each electronic medical record to a normalized patient data structure; applying the set of condition-related criteria logic rules and the at least one threshold to the plurality of normalized patient data objects to produce, for each normalized patient data object, a clinical relevance indicator indicated a degree of relevance of the clinically significant information of each patient to the condition; and generating a dashboard user interface to display a list of the plurality of patients ordered according to relevance to the condition based at least in part on the degree of relevance of the clinically significant information of each patient to the condition; where the dashboard user interface presents each normalized patient data structure and each electronic medical record associated with each patient.

Clause 16. The non-transitory computer-readable medium as recited in clause 15, where the condition is associated with at least one treatment including at least one of: Left Atrial Appendage Closure (LAAC), pulmonary artery (PA) sensor, mitral transcatheter edge-to-edge repair (TEER), aortic valve replacement (AVR), mitral valve replacement (MVR), implantable cardioverter-defibrillator (ICD), cardiac resynchronization therapy (CRTD), or atrial fibrillation (AF) ablation.

Clause 17. The non-transitory computer-readable medium as recited in clause 15, where the at least one clinical data parser includes at least one severity natural language processor configured to process echocardiogram reports and extract indications of severity of a cardiac condition.

Clause 18. The non-transitory computer-readable medium as recited in clause 15, where the at least one clinical data parser is configured to identify and extract at least one diagnostic code from the at least one diagnostic code from the plurality of electronic medical records.

Clause 19. The non-transitory computer-readable medium as recited in clause 15, where the at least one clinical data parser is configured to identify and extract at least one laboratory test result; and where the at least one threshold includes at least one threshold value associated with the at least one laboratory test result, the at least one threshold value defining relevance of the at least one laboratory test result to the condition.

Clause 20. The non-transitory computer-readable medium as recited in clause 15, where the instructions further cause at least one processor to perform steps: utilizing at least one condition-related criteria machine learning model to ingest the condition-related criteria and, based at least in part on a plurality of trained condition-related criteria machine learning model parameters, output: the at least one health data type associated with the clinically significant information for the condition, and the at least one threshold for the at least one health data type; and generating the set of logic rules based at least in part on: the at least one health data type associated with the clinically significant information for the condition, and the at least one threshold for the at least one health data type.

Publications cited throughout this document are hereby incorporated by reference in their entirety. While one or more embodiments of the present disclosure have been described, it is understood that these embodiments are illustrative only, and not restrictive, and that many modifications may become apparent to those of ordinary skill in the art, including that various embodiments of the inventive methodologies, the illustrative systems and platforms, and the illustrative devices described herein can be utilized in any combination with each other. Further still, the various steps may be carried out in any desired order (and any desired steps may be added and/or any desired steps may be eliminated).

What is claimed is:

1. A method comprising:
determining, by at least one processor, a condition-related criteria associated with diagnosis or treatment or both of a condition;
determining, by the at least one processor, a set of condition-related criteria logic rules derived from the condition-related criteria, wherein the set of condition-related criteria logic rules comprise logic-based rules that define clinically significant information for the condition;
wherein the set of condition-related criteria logic rules comprise:
at least one health data type associated with the clinically significant information for the condition, and
at least one threshold for the at least one health data type;
determining, by the at least one processor, at least one clinical data parser configured to parse data associated with the at least one health data type so as to extract the clinically significant information from the data;
inputting, by the at least one processor, a plurality of electronic medical record associated with a plurality of patients into the at least one clinical data parser to extract the clinically significant information from each electronic medical record;
generating, by the at least one processor, a plurality of normalized patient data objects for the plurality of patients by mapping the clinically significant information extracted from each electronic medical record to a normalized patient data structure;
applying, by the at least one processor, the set of condition-related criteria logic rules and the at least one threshold to the plurality of normalized patient data objects to produce, for each normalized patient data object, a clinical relevance indicator indicating a degree of relevance of the clinically significant information of each patient to the condition; and
generating, by the at least one processor, a dashboard user interface to display a list of the plurality of patients ordered according to relevance to the condition based at least in part on the degree of relevance of the clinically significant information of each patient to the condition;
wherein the dashboard user interface presents each normalized patient data structure and each electronic medical record associated with each patient.

2. The method as recited in claim 1, wherein the condition is associated with at least one treatment comprising at least one of:
Left Atrial Appendage Closure (LAAC),
pulmonary artery (PA) sensor,
mitral transcatheter edge-to-edge repair (TEER),
aortic valve replacement (AVR),
mitral valve replacement (MVR),
implantable cardioverter-defibrillator (ICD),
cardiac resynchronization therapy (CRTD), or
atrial fibrillation (AF) ablation.

3. The method as recited in claim 1, wherein the at least one clinical data parser comprises at least one severity natural language processor configured to process echocardiogram reports and extract indications of severity of a cardiac condition.

4. The method as recited in claim 1, wherein the at least one clinical data parser is configured to identify and extract at least one diagnostic code from the plurality of electronic medical records.

5. The method as recited in claim 1, wherein the at least one clinical data parser is configured to determine at least one admissions statistic associated with at least one patient of the plurality of patients based at least in part on at least one time associated with at least one entry in at least one electronic medical of the plurality of electronic medical records.

6. The method as recited in claim 1, wherein the at least one clinical data parser is configured to identify and extract at least one laboratory test result; and
wherein the at least one threshold comprises at least one threshold value associated with the at least one laboratory test result, the at least one threshold value defining relevance of the at least one laboratory test result to the condition.

7. The method as recited in claim 1, further comprising:
utilizing, by the at least one processor, at least one condition-related criteria machine learning model to ingest the condition-related criteria and, based at least in part on a plurality of trained condition-related criteria machine learning model parameters, output:
the at least one health data type associated with the clinically significant information for the condition, and
the at least one threshold for the at least one health data type; and
generating, by the at least one processor, the set of logic rules based at least in part on:
the at least one health data type associated with the clinically significant information for the condition, and
the at least one threshold for the at least one health data type.

8. A system comprising:
at least one processor which, upon execution of instructions, is configured to:
determine a condition-related criteria associated with diagnosis or treatment or both of a condition;
determine a set of condition-related criteria logic rules derived from the condition-related criteria, wherein the set of condition-related criteria logic rules comprise logic-based rules that define clinically significant information for the condition;
wherein the set of condition-related criteria logic rules comprise:
at least one health data type associated with the clinically significant information for the condition, and
at least one threshold for the at least one health data type;
determine at least one clinical data parser configured to parse data associated with the at least one health data type so as to extract the clinically significant information from the data;
input a plurality of electronic medical record associated with a plurality of patients into the at least one clinical data parser to extract the clinically significant information from each electronic medical record;
generate a plurality of normalized patient data objects for the plurality of patients by mapping the clinically significant information extracted from each electronic medical record to a normalized patient data structure;
apply the set of condition-related criteria logic rules and the at least one threshold to the plurality of normalized patient data objects to produce, for each normalized patient data object, a clinical relevance indicator indicated a degree of relevance of the clinically significant information of each patient to the condition; and
generate a dashboard user interface to display a list of the plurality of patients ordered according to relevance to the condition based at least in part on the degree of relevance of the clinically significant information of each patient to the condition;
wherein the dashboard user interface presents each normalized patient data structure and each electronic medical record associated with each patient.

9. The system as recited in claim 8, wherein the condition is associated with at least one treatment comprising at least one of:
Left Atrial Appendage Closure (LAAC),
pulmonary artery (PA) sensor,
mitral transcatheter edge-to-edge repair (TEER),
aortic valve replacement (AVR),
mitral valve replacement (MVR),
implantable cardioverter-defibrillator (ICD),
cardiac resynchronization therapy (CRTD), or
atrial fibrillation (AF) ablation.

10. The system as recited in claim 8, wherein the at least one clinical data parser comprises at least one severity natural language processor configured to process echocardiogram reports and extract indications of severity of a cardiac condition.

11. The system as recited in claim 8, wherein the at least one clinical data parser is configured to identify and extract at least one diagnostic code from the plurality of electronic medical records.

12. The system as recited in claim 8, wherein the at least one clinical data parser is configured to determine at least one admissions statistic associated with at least one patient of the plurality of patients based at least in part on at least one time associated with at least one entry in at least one electronic medical of the plurality of electronic medical records.

13. The system as recited in claim 8, wherein the at least one clinical data parser is configured to identify and extract at least one laboratory test result; and
wherein the at least one threshold comprises at least one threshold value associated with the at least one laboratory test result, the at least one threshold value defining relevance of the at least one laboratory test result to the condition.

14. The system as recited in claim 8, wherein the at least one processor is further configured to:
utilize at least one condition-related criteria machine learning model to ingest the condition-related criteria and, based at least in part on a plurality of trained condition-related criteria machine learning model parameters, output:
the at least one health data type associated with the clinically significant information for the condition, and
the at least one threshold for the at least one health data type; and
generate the set of logic rules based at least in part on:
the at least one health data type associated with the clinically significant information for the condition, and
the at least one threshold for the at least one health data type.

15. A method comprising:
obtaining, by at least one processor, a condition-related criteria associated with diagnosis or treatment or both of a condition;
inputting, by the at least one processor, a plurality of electronic medical records associated with a plurality of patients into at least one condition-related clinical data parsing pipeline configured to parse data associated with the condition so as to extract the clinically significant information from the plurality of electronic medical records;
generating, by the at least one processor, a plurality of normalized patient data objects for the plurality of patients by mapping the clinically significant information extracted from each electronic medical record to a normalized patient data structure; and
generating, by the at least one processor, a dashboard user interface to display a list of the plurality of patients ordered according to relevance to the condition based at least in part on the clinically significant information of each patient to the condition;
wherein the dashboard user interface presents each normalized patient data structure and each electronic medical record associated with each patient.

16. The method as recited in claim 15, wherein the condition is associated with at least one treatment comprising at least one of:
Left Atrial Appendage Closure (LAAC),
pulmonary artery (PA) sensor,
mitral transcatheter edge-to-edge repair (TEER),
aortic valve replacement (AVR),
mitral valve replacement (MVR),
implantable cardioverter-defibrillator (ICD),
cardiac resynchronization therapy (CRTD), or
atrial fibrillation (AF) ablation.

17. The method as recited in claim 15, wherein the at least one condition-related clinical data parsing pipeline comprises at least one severity natural language processor configured to process echocardiogram reports and extract indications of severity of a cardiac condition.

18. The method as recited in claim 15, wherein the at least one condition-related clinical data parsing pipeline is configured to identify and extract at least one diagnostic code from the at least one diagnostic code from the plurality of electronic medical records.

19. The method as recited in claim 15, wherein the at least one condition-related clinical data parsing pipeline is configured to identify and extract at least one laboratory test result; and
wherein the at least one threshold comprises at least one threshold value associated with the at least one laboratory test result, the at least one threshold value defining relevance of the at least one laboratory test result to the condition.

20. The method as recited in claim 15, further comprising:
utilizing, by the at least one processor, at least one condition-related criteria machine learning model to ingest the condition-related criteria and, based at least in part on a plurality of trained condition-related criteria machine learning model parameters, output:
at least one health data type associated with the clinically significant information for the condition, and
at least one threshold for the at least one health data type; and
generating, by the at least one processor, a set of logic rules based at least in part on:
the at least one health data type associated with the clinically significant information for the condition, and
the at least one threshold for the at least one health data type.
